(19) United States
(12) Patent Application Publication
(10) Pub. No.: US 2007/0259875 A1
(43) Pub. Date: Nov. 8, 2007

(54) Triaryl substituted imidazole derivatives and taste-inhibiting uses thereof

(76) Inventors: Karnail S. Atwal, Pennington, NJ (US); Anita B. Atwal, Pennington, NJ (US); Robert W. Bryant, Princeton, NJ (US); Seunghun Paul Lee, Newton, PA (US); Roy Kyle Palmer, Cranbury, NJ (US); Rok Cerne, Lawrenceville, NJ (US)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 11/797,082

(22) Filed: Apr. 30, 2007

Related U.S. Application Data
(60) Provisional application No. 60/796,235, filed on Apr. 28, 2006.

(51) Int. Cl. A61K 31/53; A61K 31/501; A61K 31/497; A61K 31/506; A61K 31/4439; A61K 31/4164

(52) U.S. Cl. 514/241; 514/252.05; 514/255.05; 514/256; 514/396; 514/341

(57) ABSTRACT

The present invention is directed to compositions containing and methods of using a compound having the formula: 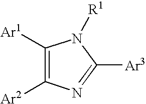
wherein R¹, Ar¹, Ar², and Ar³ are defined herein. The compounds of the present invention are useful as inhibitors of certain taste perceptions and functions. The invention is also directed to compositions comprising a compound of the above formula.

### CROSS-REFERENCE TO RELATED APPLICATIONS

This application claims the benefit of the filing date of U.S. Patent Appl. No. 60/796,235, filed Apr. 28, 2006, which is incorporated by reference herein in its entirety.

### BACKGROUND OF THE INVENTION

1. Field of the Invention

The present invention relates to the use of compounds of Formula I for inhibiting certain taste functions and perceptions and related uses. The invention is also directed to, among other things, compositions comprising a compound of Formula I that can be used in pharmaceutical, food, and other compositions to inhibit certain taste functions and perceptions.

2. Background

Taste perception plays a critical role in the nutritional status and survival of both lower and higher animals (Margolskee, R. F., J. Biol. Chem. 277:1-4 (2002); Avenet, P. and Lindemann, B. J., Membrane Biol. 112:1-8 (1989)). The ability to taste has significance beyond providing people with pleasurable culinary experiences. For example, the ability to taste allows us to identify tainted or spoiled foods, and provides satisfying responses that can be proportionate to caloric or nutritive value.

Although taste perception is a vital function, sometimes it is useful to modify certain tastes. For example, many active pharmaceutical ingredients of medicines produce an undesirable taste, such as a bitter taste. The same holds true for some compounds that are ingredients or additives in nutriceuticals, foods, dental hygiene products and cosmetics. Masking or inhibiting the production of an undesirable taste by these products can lead to improved acceptance by the patient or consumer.

Traditionally, sweeteners and flavorants have been used to mask the bitter taste of pharmaceuticals. The sweetener or flavorant is known to activate taste pathways (other than the pathway producing the undesirable taste), and at sufficiently high concentration, can mask the bitter taste of the pharmaceutical. However, this approach has proved ineffective at masking the taste of very bitter compounds. Microencapsulation in a cellulose derivative has also been used to mask the bitter taste of some compounds. However, this approach prevents rapid oral absorption of the pharmaceutical.

Thus the presently available methods for inhibiting, altering, or masking unwanted tastes are insufficient. There exists a need for compounds that can effectively inhibit an unwanted taste.

Taste also plays a role the appetite for food. Studies have shown increased food intake as palatability increased. Sorensen, et al., Int. J. Obes. Relat. Metab. Disord. 27(10):1152-66 (2003). Conversely, certain drugs, such as antihypertensives and antihyperlipidemics, have been reported to produce untoward alterations in taste and can result in decreased food intake. Doty, et al., J Hypertens. 21(10):1805-13 (2003). Taste impairment associated with radiation treatments for head and neck cancer has been considered to be one of the factors associated with reduced appetite and altered patterns of food intake in these patients. Vissink, et al., Crit. Rev. Oral Biol. Med. 14(3):213-25 (2003). Decreased food consumption has also been correlated with loss of taste sensations in the elderly. Shiffman, S. S., J. Am. Med. Ass'n 278(16):1357-1362 (1997).

Much research has been done in an attempt to find safe and effective means for decreasing food intake in people in need of weight reduction. A number of agents have been developed and marketed to reduce appetite and food intake, such as amphetamine derivatives and fenfluramine. However, many have dangerous side effects. More selective approaches, e.g., neuro-regulation via peptide mimetics/antagonists, are still in developmental phases.

Therefore, there exists a need for compounds that can effectively decrease palatability of food, without dangerous adverse effects, to reduce food intake.

### BRIEF SUMMARY OF THE INVENTION

The present invention provides methods and compositions for inhibiting, altering, or masking unwanted tastes. The present invention also provides methods and compositions for inhibiting, masking or altering tastes to decrease palatability of food and lead to reduced food intake

The compositions and methods of the invention use a triaryl substituted imidazole of Formula I: 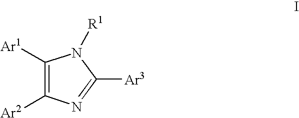
or a pharmaceutically acceptable salt thereof; wherein R¹ is hydrogen, unsubstituted alkyl, or unsubstituted arylalkyl; Ar¹ and Ar² are independently phenyl or a heteroaryl, either of which is optionally substituted as described herein below; and Ar³ is phenyl or a heteroaryl, either of which is optionally substituted as described herein below.

In some embodiments of the present invention, Ar¹ and Ar² are independently selected from the group consisting of phenyl, pyridyl, pyrimidinyl, pyridazinyl, pyrimidinyl, pyrazinyl, and triazinyl, and N-oxides thereof, each of which is optionally substituted as described herein below.

In some embodiments of the present invention, Ar¹ and Ar² are independently selected from the group consisting of unsubstituted phenyl, methylphenyl, methoxyphenyl, halophenyl, cyanophenyl, carboxyphenyl, aminophenyl, and hydroxyphenyl, wherein the phenyl substitution can be at any one or more of the ortho-, meta-, and para-positions.

In some embodiments of the present invention, Ar¹ and Ar² are both unsubstituted phenyl, or alternatively, Ar¹ and Ar² are both unsubstituted: C-attached pyridyl, C-attached pyridazinyl, C-attached pyrimidinyl, C-attached pyrazinyl, C-attached triazinyl, and N-oxides thereof.

In some embodiments of the present invention, Ar³ is selected from the group consisting of phenyl, pyridyl, pyrimidinyl, pyridazinyl, pyrimidinyl, pyrazinyl, and triazinyl, and N-oxides thereof, each of which can be optionally substituted as described herein below.

In some embodiments of the present invention, Ar³ is phenyl, pyrid-2-yl, pyrid-3-yl, pyrid-4-yl, pyridazin-3-yl, pyridazin-4-yl, pyrimidin-2-yl, pyrimidin-4-yl, pyrimidin-5-yl, pyrazin-2-yl, 1,2,3-triazin-4-yl, 1,2,3-triazin-5-yl, or 1,3,5-triazin-2-yl, any of which is optionally substituted as described herein below.

In some embodiments of the present invention, Ar³ is a C-attached pyridyl, C attached pyridazinyl, C-attached pyrimidinyl, C-attached pyrazinyl, C-attached triazinyl, and N-oxides thereof, any of which is optionally substituted as described herein below.

In some embodiments, R¹ is hydrogen, an unsubstituted C₁₋₄ alkyl, or an unsubstituted aryl(C₁₋₄)alkyl; Ar¹ and Ar² are both unsubstituted phenyl; and Ar³ is phenyl, substituted by one to three substituents independently selected from the group consisting of carboxy, alkoxycarbonyl, hydroxy, hydroxyalkyl, amino, alkoxycarbonylamino, cyano, alkylsulfonylaminoalkyl, and nitro.

In some embodiments, Ar³ is selected from the group consisting of phenyl, C-attached pyridyl, C-attached pyrimidyl, and C-attached pyridazinyl, optionally substituted with one or more nitro, halo, cyano, carboxyl, amino, hydroxyl, alkyl, and cyanoalkyl substituents in any one or more of the ortho-, meta-, and para-positions.

In some embodiments, Ar³ is phenyl, substituted in the para-position by a carboxy, alkoxycarbonyl, hydroxyalkyl, hydroxy, amino, alkoxycarbonylamino, cyano, alkylsulfonylaminoalkyl, or nitro.

In some embodiments, the compound of Formula I is one of:
methyl4-(4,5-diphenyl-1H-imidazol-2-yl)benzoate;
[4-(4,5-diphenyl-1H-imidazol-2-yl)phenyl]methanol;
4-(4,5-diphenyl-1H-imidazol-2-yl)aniline;
4-(4,5-diphenyl-1H-imidazol-2-yl)phenol;
methyl4-(4,5-diphenyl-1H-imidazol-2-yl)phenylcarbamate;
N-[4-(4,5-diphenyl-1H-imidazol-2-yl)phenyl]acetamide;
4-(4,5-diphenyl-1H-imidazol-2-yl)benzonitrile;
N-[4-(4,5-diphenyl-1H-imidazol-2-yl)benzyl]methanesulfonamide;
4-(4,5-diphenyl-1H-imidazol-2-yl)benzoic acid; and
2-(4-nitro-phenyl)-4,5-diphenyl-1H-imidazole;
   and pharmaceutically acceptable salts thereof.

The present invention is directed to a method of inhibiting a taste modulating protein, the method comprising contacting the taste modulating protein with a compound of Formula I, or a pharmaceutically acceptable salt thereof.

In some embodiments, the taste modulating protein is a non-human TRPM5 protein. Such proteins suitable for inhibiting with a compound of Formula I include, but are not limited to, those from a cow, horse, sheep, pig, cat, dog, rabbit, or monkey. In some embodiments, the species is human. In some embodiments, the taste modulating protein is in vitro.

The present invention is also directed to a method of masking a taste, the method comprising administering to a subject in need of taste masking one or more compounds of Formula I or a pharmaceutically acceptable salt thereof. A subject in need of taste masking can be a human. A taste in need of masking can be a bitter taste.

The present invention is also directed to a method of inhibiting the depolarization of a taste receptor cell, the method comprising contacting the taste receptor cell with a compound of Formula I or a pharmaceutically acceptable salt thereof.

In some embodiments of the present invention, the compound of Formula I is administered in an amount sufficient to inhibit a taste-modulating protein, mask a taste, or inhibit the depolarization of a taste-receptor cell by about 10% to about 95%.

The present invention is also directed to a method of decreasing the palatability of food in a subject, comprising administering to a subject in need thereof one or more compounds of Formula I, or a pharmaceutically acceptable salt thereof.

In some embodiments, the palatability of food can be reduced in a subject by administering a compound of Formula I using a solid dosage form, an orally disintegrating dosage form, a liquid dosage form, a suspension, an aerosol composition, a buccal patch, a surgical implant, a depot injection, or intravenously. In some embodiments, a compound of Formula I can be administered to a subject immediately prior to a meal. A subject in need of such treatment can suffer from compulsive overeating disorder, bulimia, binge eating disorder, obesity, over-eating, or eating without care for stopping.

In some embodiments, a reduction in the palatability of food in a subject can result in a decreased caloric intake in a subject. A subject in need of such treatment can suffer from, type-II diabetes, or other diabetes related disorders, or alternatively, have a body mass index of at least 30. In some embodiments, administering a compound of Formula I to a subject in need thereof can decrease the caloric intake of the subject by at least about 10%.

In some embodiments, a taste in need of masking arises from a component of a pharmaceutical, veterinary, food, cosmetic, or dental hygienic composition. The method of taste masking can comprise adding a compound of Formula I to the pharmaceutical, veterinary, food, cosmetic, or dental hygienic composition in need of taste masking, or alternatively, administering a compound of Formula I as a separate composition to a subject in need of such treatment.

Compositions having a taste in need of masking can contain biologically active agents in addition to compounds of Formula I. Biologically active agent suitable for use with the present invention include, but are not limited to, analgesics, anesthetics, anorexiants, appetite depressants, antacids, antiasthmatics, antidiuretics, antipyretics, antihistamines, anticholinergics, antidiarrheals, antitussives, antinauseants, antiarrhythmics, antimicrobials, antibacterials, antifungals, antivirals, anti-inflammatory agents, agents active against flatulence, antimigraine agents, beta-receptor blockers, bronchodilators, psychopharmacological agents, spasmolytics, sedatives, antihyperkinetics, tranquilizers, decongestants, demulcents, agents for alcohol withdrawal, antitussives, fluorine supplements, laxatives, local antibiotics, corticosteroid supplements, agents against goiter formation, antiepileptics, agents against dehydration, antiseptics, NSAIDs, H₂-receptor antagonists, nutritional supplements, gastrointestinal active agents, alkaloids, supplements for trace elements, ion-exchange resins, cholesterol-depressant agents, lipid-lowering agents, expectorants, and combinations thereof.

In some embodiments of the present invention, a compound of Formula I can be administered as a pharmaceutical, veterinary, food, cosmetic, or dental hygienic composition to a subject in need thereof. A compound of Formula I can be present in a pharmaceutical, veterinary, food, cosmetic, or dental hygienic composition of the present invention in an amount of about 0.01 mg to about 100 mg.

The present invention is also directed to a pharmaceutical composition comprising one or more pharmaceutically acceptable carriers, and one or more compounds of Formula I or a pharmaceutically acceptable salt thereof.

The present invention is also directed to a method of preparing an improved pharmaceutical composition, wherein the improvement comprises adding to a pharmaceutical composition one or more compounds of Formula I, or a pharmaceutically acceptable salt thereof. In some embodiments, the pharmaceutical composition of the present invention is a solid dosage form, an orally disintegrating dosage form, a liquid dosage form, a suspension, an aerosol composition, a buccal patch, a surgical implant, a depot injection, or an intravenous solution.

The present invention is also directed to a food composition comprising one or more food ingredients and one or more compounds of Formula I, or a pharmaceutically acceptable salt thereof.

The present invention is also directed to a method of preparing an improved food composition, wherein the improvement comprises adding to a food composition one or more compounds of Formula I, or a pharmaceutically acceptable salt thereof.

In some embodiments, the food composition of the present invention is suitable for human consumption, or alternatively, for animal consumption.

In some embodiments, the food composition of the present invention is a liquid, or alternatively, a solid. Food compositions of or for use with the present invention include, but are not limited to, citrus fruits, vegetables, seasoning or flavoring materials, soybean compositions, fish, meats and processed meats, dairy, breads and cakes, and confectioneries.

The present invention is also directed to a cosmetic composition comprising one or more dental cosmetic ingredients and a compound of Formula I, or a pharmaceutically acceptable salt thereof.

The present invention is also directed to a method of preparing an improved cosmetic composition, wherein the improvement comprises adding to a cosmetic composition one or more compounds of Formula I, or a pharmaceutically acceptable salt thereof. Cosmetic compositions of or for use with the present invention include, but are not limited to, face cream, lipstick, lip gloss, and lip balm.

The present invention is also directed to a dental hygienic composition comprising one or more dental hygienic ingredients and a compound of Formula I, or a pharmaceutically acceptable salt thereof.

The present invention is also directed to a method of preparing an improved dental hygienic composition, wherein the improvement comprises adding to a dental hygienic composition one or more compounds of Formula I, or a pharmaceutically acceptable salt thereof. Dental hygienic compositions of or for use with the present invention include, but are not limited to, toothpaste, mouthwash, plaque rinse, a teeth-whitening composition, and mouth spray.

In some embodiments, a compound of Formula I is present in a pharmaceutical, veterinary, food, cosmetic, or dental hygienic composition of the present invention in a concentration of about 10⁻⁷ to about 10⁻³ by mole, or about 10⁻⁶ to about 10⁻⁴ by mole of the unit dose of the pharmaceutical, veterinary, food, cosmetic, or dental hygienic composition. In some embodiments, a compound of Formula I is present in a pharmaceutical, veterinary, food, cosmetic, or dental hygienic composition of the present invention in a concentration of about 10⁻⁶ to about 10⁻², or about 10⁻⁵ to about 10⁻³ by weight, of the pharmaceutical, veterinary, food, cosmetic, or dental hygienic composition.

These and additional aspects of the present invention are described in detail below.

### BRIEF DESCRIPTION OF THE DRAWINGS/FIGURES

The accompanying drawings, which are incorporated herein and form a part of the specification, serve to explain the principles of the invention and to enable a person skilled in the pertinent art to make and use the invention.

FIG. 1 illustrates the generation of a response in the taste modulating protein TRPM5. The response changes cell membrane potential, which can be detected, e.g., using fluorescent dyes and a fluorescent imaging plate reader (FLIPR).

FIG. 2(A) and 2(B) illustrate the TRPM5-dependent fluorescent signal in HEK293 cells. The experimental details are explained in EXAMPLE 11.

### DETAILED DESCRIPTION OF THE INVENTION

The present invention provides compounds and compositions that are useful, for example, for inhibiting the activity of a taste modulating protein. Other aspects of the present invention are described in detail herein.

The methods and composition of the present invention can also include a pharmaceutically acceptable salt of a compound of Formula I. The term pharmaceutically acceptable salt refers to an acid- and/or base-addition salt of a compound of Formula I. Acid-addition salts can be formed by adding an appropriate acid to the compound of Formula I. Base-addition salts can be formed by adding an appropriate base to the compound of Formula I. The acid or base does not substantially degrade, decompose, or destroy the compound of Formula I. Examples of suitable pharmaceutically acceptable salts include hydrochloride, hydrobromide, acetate, furmate, maleate, oxalate, and succinate salts. Other suitable salts include sodium, potassium, carbonate, and tromethamine salts.

The present invention is considered to encompass the use of stereoisomers as well as optical isomers, e.g., mixtures of enantiomers as well as individual enantiomers and diastereomers, which arise as a consequence of structural asymmetry in selected compounds of the present series. It is further understood that the present invention encompasses the use of tautomers of a compound of Formula I. Tautomers are well-known in the art and include keto-enol tautomers.

The compounds of Formula I can also be solvated, including hydrated. Hydration can occur during manufacturing of the compounds or compositions comprising the compounds, or the hydration can occur over time due to the hygroscopic nature of the compounds.

Certain compounds within the scope of Formula I can be derivatives referred to as “prodrugs.” The expression “prodrug” denotes a derivative of a known direct acting agent, wherein the derivative has therapeutic value that can be similar to, greater than, or less than that of the agent. Generally, the prodrug is transformed into the active agent by an enzymatic or chemical process when delivered to the subject, cell, or test media. In certain instances, prodrugs are derivatives of the compounds of the invention which have metabolically cleavable groups and become by solvolysis or under physiological conditions the compounds of the invention which are pharmaceutically active in vivo. For example, ester derivatives of compounds of this invention are often active in vivo, but not in vitro. Other derivatives of the compounds of this invention have activity in both their acid and acid derivative forms, but the acid derivative form often offers advantages of solubility, tissue compatibility, or delayed release in the mammalian organism (see, Bundgard, H., Design of Prodrugs, pp. 7-9, 21-24, Elsevier, Amsterdam (1985)). Prodrugs include acid derivatives well known to practitioners of the art, such as, for example, esters prepared by reaction of the parent acid with a suitable alcohol, or amides prepared by reaction of the parent acid compound with an amine. Simple aliphatic or aromatic esters derived from acidic groups pendent on the compounds of this invention are preferred prodrugs. In some cases, it is desirable to prepare double ester type prodrugs such as (acyloxy) alkyl esters or [(alkoxycarbonyl)oxy]alkyl esters.

When any variable occurs more than one time in any constituent or in Formula I, its definition on each occurrence is independent of its definition at every other occurrence, unless otherwise indicated. Also, combinations of substituents and/or variables are permissible only if such combinations result in stable compounds.

Unless otherwise indicated, the term “alkyl” or “alk” as used herein alone or as part of another group includes both straight and branched chain hydrocarbons, containing 1 to 20 carbons, preferably 1 to 10 carbons, more preferably 1 to 8 carbons, such as methyl, ethyl, propyl, isopropyl, butyl, t-butyl, isobutyl, pentyl, hexyl, isohexyl, heptyl, 4,4-dimethylpentyl, octyl, 2,2,4-trimethylpentyl, nonyl, decyl, undecyl, dodecyl, and the like. Lower alkyl refers to such groups containing 1-6 carbon atoms. Unless specified otherwise, an alkyl group may be optionally substituted with 1 or more ‘alkyl substituents’ which may be the same or different at each occurrence. These substituents may occur at any place and in any combination that provides a stable compound.

As used herein, “optionally substituted” refers to substitution with one or more of the following substituents, which may be the same or different at each occurrence, and which can occur at any place and in any combination that provides a stable compound.

These ‘optional substituents’ can be:

halogen;

nitro;

cyano;

OR₂₂;

alkyl which may be substituted with one or more occurrences of R₂₃;

alkenyl which may be substituted with one or more occurrences of R₂₃;

alkynyl which may be substituted with one or more occurrences of R₂₃;

cycloalkyl which may be substituted with one or more occurrences of R₂₃;

aryl which may be substituted with one or more occurrences of R₂₃;

heterocyclo which may be substituted with one or more occurrences of R₂₃;

SR₂₂;

SO₂R₂₂;

COOR₂₂;

C(O)R₂₂;

CONR₂₄R₂₅;

SO₂NR₂₄R₂₅;

SO₂N(H)C(O)R₂₂;

SO₂N(H)CO₂R₂₂, wherein R₂₂ is not H;

NR₂₄R₂₅;

N(R₂₄)SO₂R₂₅;

N(R₂₄)C(O)ₘR₂₅ (wherein m=1,2);

N(R₂₄)C(O)NR₂₅R₂₆;

N(R₂₄)SO₂NR₂₅R₂₆;

OC(O)R₂₂;

OC(O)OR₂₂;

OC(O)NR₂₅R₂₆;

C(O)N(H)SO₂NR₂₅R₂₆;

C(O)N(H)SO₂R₂₅;

oxo (or keto, i.e., ═O);

thioxo (i.e., ═S);

imino (i.e., ═NR²⁷);

NR₂₇—C(═NR₂₈)R₂₉;

NR₂₇—C(═NR₂₈)NR₂₉R₃₀;

C(═NR₂₇)NR₂₈R₂₉;

OC(═NR₂₇)NR₂₈R₂₉;

OC(═NR₂₇)R₂₈;

C(═NR₂₇)R₂₈;

C(═NR₂₇)OR₂₂;

wherein R₂₂ is selected from H, C₁-C₈ alkyl, C₂-C₈ alkenyl, C₂-C₈ alkynyl, C₃-C₈ cycloalkyl, C₆-C₁₀ aryl, or C₁-C₉ heterocyclo each of which may be substituted with 1 to 3 independent occurrences of R₂₃; and

wherein R₂₄, R₂₅, and R₂₆ are selected from C₁-C₈ alkyl, C₂-C₈ alkenyl, C₂-C₈ alkynyl, C₃-C₈ cycloalkyl, C₆-C₁₀ aryl, or C₁-C₉ heterocyclo each of which may be substituted with 1 to 3 independent occurrences of R₂₃, or R₂₄ and R₂₅, or R₂₄ and R₂₆ or R₂₅ and R₂₆ may be joined by an alkylene or an alkenylene chain to form a 5- to 8-membered heterocyclo ring which is defined as for heterocyclo wherein the substituents may be one or more occurrences of R₂₃.

R₂₇, R₂₈, R₂₉, or R₃₀ are independently selected from H, nitro, cyano, OH, O(C₁-C₆ alkyl), C(O)R₂₂, C(O)NR₂₄R₂₅, CO₂R₂₂ (with the proviso that R₂₂ is not H), SO₂R₂₂, SO₂NR₂₄R₂₅, C₁-C₈ alkyl, C₂-C₈ alkenyl, C₂-C₈ alkynyl, C₃-C₈ cycloalkyl, C₆-C₁₀ aryl, or C₁-C₉ heterocyclo or R₂₇ and R₂₈ or R₂₇ and R₂₉ or R₂₇ and R₃₀ or R₂₈ and R₂₉ or R₂₈ and R₃₀ or R₂₉ and R₃₀ may be joined by an alkylene or alkenylene chain to form a 5- to 8-membered ring that may be optionally substituted with one or more occurrences of R₂₃.

R₂₃ is selected from:

halogen;

nitro;

cyano;

OR₃₁;

alkyl optionally substituted with halogen;

cycloalkyl optionally substituted with halogen;

aryl optionally substituted with halogen, hydroxy, nitro, methoxy, trifluoromethyl, cyano, carbomethoxy, CONH₂, and CHO;

heterocyclo optionally substituted with halogen, hydroxy, nitro, methoxy, trifluoromethyl, cyano, carbomethoxy, CONH₂, and CHO,

SR₃₁;

CO₂R₃₁;

C(O)R₃₁;

CONR₃₂R₃₃;

SO₂NR₃₂R₃₃;

NR₃₂R₃₃;

N(R₃₂)SO₂R₃₃;

N(R₃₂)C(O)ₘR₃₃ (m=1, 2);

N(R₃₂)C(O)NR₃₃R₃₄;

N(R₃₂)SO₂NR₃₃R₃₄;

OC(O)R₃₁;

OC(O)OR₃₁;

SO₂R₃₁;

SO₂N(H)C(O)R₃₁;

SO₂N(H)CO₂R₃₁ wherein R₃₁ is not H;

C(O)N(H)SO₂NR₃₂R₃₃;

C(O)N(H)SO₂R₃₁;

OC(O)NR₃₂R₃₃;

NR₃₅—C(═NR₃₆)R₃₇;

NR₃₅—C(═NR₃₆)OR₃₁;

NR₃₅—C(═NR₃₆)NR₃₇R₃₈;

C(═NR₃₅)NR₃₆R₃₇;

OC(═NR₃₅)R₃₆;

OC(═NR₃₅)NR₃₆R₃₇; and

C(═NR₃₅)OR₃₁;

wherein R₃₁ is selected from unsubstituted alkyl, alkenyl, unsubstituted alkynyl, unsubstituted cycloalkyl, unsubstituted aryl, unsubstituted heterocyclo;

wherein R₃₂, R₃₃ and R₃₄ are selected from unsubstituted alkyl, unsubstituted alkenyl, unsubstituted alkynyl, unsubstituted cycloalkyl, unsubstituted aryl, unsubstituted heterocyclo, or R₃₂ and R₃₃ or R₃₂ and R₃₄ or R₃₃ and R₃₄ may be joined by an unsubstituted alkylene or unsubstituted alkenylene chain to form a 5- to 8-membered unsubstituted heterocyclo ring; and

wherein R₃₅, R₃₆, R₃₇, R₃₈ are selected from nitro, cyano, unsubstituted alkyl, unsubstituted alkenyl, unsubstituted alkynyl, unsubstituted cycloalkyl, unsubstituted aryl, unsubstituted heterocyclo, or R₃₅ and R₃₆, or R₃₅ and R₃₇ or R₃₅ and R₃₈ or R₃₆ and R₃₇ or R₃₆ and R₃₈ or R₃₇ and R₃₈ may be joined by an unsubstituted alkylene chain or unsubstituted alkenylene chain to form a 5- to 8-membered unsubstituted heterocyclo ring.

Unless otherwise indicated, the term “cycloalkyl” as employed herein alone or as part of another group includes saturated or partially unsaturated (i.e., containing one or more carbon-carbon double bonds) cyclic hydrocarbon groups containing 1 to 3 rings, containing a total of 3 to 20 carbons forming the ring(s), preferably 3 to 10 carbons, forming the ring. Polycyclic systems may contain fused or bridged rings or both. In addition, the cycloalkyl group may be fused to 1 or 2 aryl rings. Examples include cyclopropyl, cyclobutyl, cyclopentyl, cyclohexyl, cycloheptyl, cyclooctyl, cyclodecyl and cyclododecyl, cyclohexenyl,

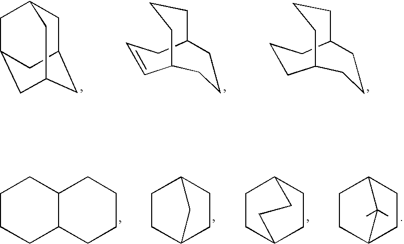

Cycloalkyl groups may be optionally substituted with 1 or more “cycloalkyl substituents” which may be the same or different at each occurrence. These optional substituents may occur at any place in any combination that provides a stable compound. These substituents may be any of the optional substituents as defined above.

The term “alkanoyl” as used herein alone or as part of another group refers to alkyl linked to a carbonyl group.

Unless otherwise indicated, the term “alkenyl” as used herein by itself or as part of another group refers to straight or branched chain radicals of 2 to 20 carbons, preferably 2 to 12 carbons, and more preferably 2 to 8 such as vinyl, 2-propenyl, 3-butenyl, 2-butenyl, 4-pentenyl, 3-pentenyl, 2-hexenyl, 3-hexenyl, 2-heptenyl, 3-heptenyl, 4-heptenyl, 3-octenyl, 3-nonenyl, 4-decenyl, 3-undecenyl, 4-dodecenyl, 4,8,12-tetradecatrienyl, and the like. Lower alkenyl refers to such groups containing 2-6 carbon atoms. Alkenyl groups may be optionally substituted with 1 or more ‘alkenyl substituents’ which may be the same or different at each occurrence. These optional substituents may occur at any place in any combination that provides a stable compound. These substituents may be any of the optional substituents as defined above.

Unless otherwise indicated, the term “lower alkynyl” or “alkynyl” as used herein by itself or as part of another group refers to straight or branched chain radicals of 2 to 20 carbons, preferably 2 to 12 carbons and more preferably 2 to 8 carbons such as 2-propynyl, 3-butynyl, 2-butynyl, 4-pentynyl, 3-pentynyl, 2-hexynyl, 3-hexynyl, 2-heptynyl, 3-heptynyl, 4-heptynyl, 3-octynyl, 3-nonynyl, 4-decynyl, 3-undecynyl, 4-dodecynyl and the like. Lower alkynyl refers to such groups containing 1-6 carbon atoms. Alkynyl groups may be optionally substituted with 1 or more ‘alkynyl substituents’ which may be the same or different at each occurrence. These substituents may occur at any place in any combination that provides a stable compound. These substituents may be any of those as defined above.

Where alkyl groups as defined above have single bonds for attachment to other groups at two different carbon atoms, they are termed “alkylene” groups and may optionally be substituted as defined above for “alkyl”.

Where alkenyl groups as defined above and alkynyl groups as defined above, respectively, have single bonds for attachment at two different carbon atoms, they are termed “alkenylene groups” and “alkynylene groups”, respectively, and may optionally be substituted as defined above for “alkenyl” and “alkynyl”.

Unless otherwise indicated, the term “aryl” as employed herein alone or as part of another group refers to monocyclic and bicyclic aromatic groups containing 6 to 10 carbons in the ring portion (such as phenyl or naphthyl including 1-naphthyl and 2-naphthyl) and may optionally include one to three additional rings fused to a carbocyclic ring. Aryl groups may be substituted with 1 or more ‘aryl substituents’ which may be the same or different at each occurrence. These substituents may occur at any place in any combination that provides a stable compound. These substituents may be any of the substituents as defined above.

Unless otherwise indicated, the term “alkylaryl” as employed herein alone or as part of another group, refers to an aryl group, as defined above, having an alkyl substituent, as defined above. Similarly, unless otherwise indicated, the terms “aralkyl” and “arylalkyl” as employed herein alone or as part of another group, refer to an alkyl group as defined above having an aryl substituent.

Unless otherwise indicated, the term “lower alkoxy”, “alkoxy”, “aryloxy”, “aralkoxy” or “heterocycloalkoxy” as employed herein alone or as part of another group includes any of the above alkyl, aralkyl, aryl, or heterocyclo groups linked to an oxygen atom.

Unless otherwise indicated, the term “acyl” as employed herein by itself or part of another group, as defined herein, refers to an organic radical linked to a carbonyl 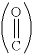
group; examples of acyl groups include any of the R¹ groups attached to a carbonyl, such as alkanoyl, alkenoyl, aroyl, aralkanoyl, heteroaroyl, cycloalkanoyl, heterocycloalkanoyl and the like.

Unless otherwise indicated, the term “heterocyclo” as used herein alone or as part of another group refers to a monocyclic or multicyclic ring system wherein one or more of the ring atoms are elements other than carbon. Preferred systems have 1 to 4 of the atoms independently selected from N, O or S. The ring system may be unsaturated, partially saturated, fully saturated or aromatic. Heterocyclo groups containing more than one ring may be fused or bridged. Heteroatoms may be optionally oxidized. Attachment may be through any available atom in the ring system. Exemplary heterocyclo (or heteraryl) groups suitable for use with the present invention include: pyridyl, pyrimidinyl, pyridazinyl, pyrimidinyl, pyrazinyl, and triazinyl, and N-oxides thereof, which may be substituted with one or more optional substituents as described above. Heterocyclo groups may be optionally substituted with 1 or more ‘heterocyclo substituents’ which may be the same or different at each occurrence. These optional substituents may occur at any place in any combination that provides a stable compound. These substituents may be any of the optional substituents as defined above.

Unless otherwise indicated, the term “heteroaryl” as used herein alone or as part of another group refers to heterocyclo groups as defined above, wherein the ring system is aromatic.

As defined above, alkyl, alkenyl, alkynyl, cycloalkyl, and heterocyclo groups may be attached through one or more single bonds to one or more attachment atoms. In addition, these groups may be attached by double bonds to attachment atoms, and these groups may be referred to as ‘alkylidene’, ‘alkenylidene’, ‘alkynylidene’, ‘cycloalkylidene’ or ‘heterocyclidene’ groups. Examples include methylidene (═CH₂), ethylidene (═CHCH₃), ethenylidene (═C═CH₂), cyclohexylidene 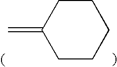
and 2-pyranylidene 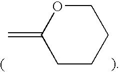
These groups may be substituted as described above for alkyl, alkenyl, alkynyl, cycloalkyl, and heterocyclo.

All stereoisomers of the compounds of the instant invention are contemplated, either in admixture or in pure or substantially pure form. The compounds of the present invention can have asymmetric centers at any of the carbon atoms including any one or the R substituents. Consequently, compounds of Formula I can exist in enantiomeric or diastereomeric forms or in mixtures thereof. The processes for preparation can utilize racemates, enantiomers or diastereomers as starting materials. When diastereomeric or enantiomeric products are prepared, they can be separated by conventional methods for example, chromatographic or fractional crystallization.

The compounds of the present invention can have asymmetric centers at certain of the nitrogen or sulfur atoms. Consequently, these isomers or mixtures thereof are part of the present invention.

The compounds of the present invention may also display other instances of chirality, such as atropoisomerism. Thus, these isomers or mixtures thereof are part of the invention.

The compounds of the present invention may also contain varying amounts of isotopes of carbon, hydrogen, nitrogen, oxygen, sulfur, halogen, etc.; such as ¹³C, ¹⁴C, deuterium, tritium, ¹⁵N, ¹⁸O, ¹²⁸I, etc. Some of the isotopic content is naturally occurring, but the compounds of the present invention may be enriched or depleted in one or more of these. Thus, these isotopes or mixtures thereof are part of the invention.

Although detailed definitions have not been provided for every term used above, each term is understood by one of ordinary skill in the art.

Methods of Use

The present invention is directed to a method of inhibiting a taste modulating protein, the method comprising contacting the taste modulating protein with a compound of Formula I, described above. Such inhibition can be in vitro or in vivo.

The amount of the compound of Formula I, or any of the specific subgroups, subclasses, or specific compounds described above, used to inhibit the taste modulating protein may not necessarily be the same when used in vivo compared to in vitro. Factors such as pharmacokinetics and pharmacodynamics of the particular compound can require that a larger or smaller amount of the compound of Formula I, or any of the specific subgroups, subclasses, or specific compounds described above, be used when inhibiting a taste modulating protein in vivo. Accordingly, one aspect of the present invention is a method of inhibiting a taste modulating protein, comprising contacting the taste modulating protein with a compound of Formula I, or any of the specific subgroups, subclasses, or specific compounds described above. In some embodiments of this aspect of the present invention, the method comprises contacting a cell with a compound of Formula I, or any of the specific subgroups, subclasses, or specific compounds described above, wherein the cell expresses the taste modulating protein. In some embodiments of the present invention, the method comprises administering a compound of Formula I, or any of the specific subgroups, subclasses, or specific compounds described above, to a subject in an amount sufficient to inhibit a taste modulating protein, wherein the subject has or expresses the taste modulating protein. Furthermore, when administered orally, the compound of Formula I can be dispersed or diluted by saliva.

As used herein, the term “inhibiting” and grammatical variants thereof refers to interfering with the normal activity of. For example, inhibiting a taste modulating protein means interfering with the normal activity of a taste modulating protein. Inhibiting includes, but is not necessarily limited to, modulating, modifying, inactivating, and the like.

By way of example, the present invention is directed to a method of inhibiting a taste modulating protein, comprising contacting the taste modulating protein with a compound of Formula I, or any of the specific subclasses and specific compounds listed above, and inhibiting the taste modulating protein by at least about 10%, 20%, 30%, 40%, 50%, 60%, 70%, 80%, 90%, or 95%, or by about 10% to about 95%, about 25% to about 95%, about 50% to about 95%, about 60% to about 95%, about 70% to about 95%, about 80% to about 95%, about 90% to about 95%, about 25% to about 80%, or about 50% to about 80%.

Additionally, the present invention is directed to a method of inhibiting a taste modulating protein, comprising contacting the taste modulating protein with a compound of Formula I, or any of the specific subclasses and specific compounds listed above, and inhibiting the taste modulating protein by at least about 10%, 20%, 30%, 40%, 50%, 60%, 70%, 80%, 90%, or 95%, or by about 10% to about 95%, about 25% to about 95%, about 50% to about 95%, about 60% to about 95%, about 70% to about 95%, about 80% to about 95%, about 90% to about 95%, about 25% to about 80%, or about 50% to about 80%, wherein the taste modulating protein is a naturally occurring taste modulating protein.

Any amount of the compound of Formula I that provides the desired degree of inhibition can be used. For example, a compound of Formula I can be used at a concentration of about 0.1 μM to about 1,000 μM to inhibit a taste modulating protein. Alternatively, concentrations of about 1, 10 or 100 μM of a compound of Formula I can be used to inhibit a taste modulating protein. In some embodiments, a single dose or two to four divided daily doses, provided on a basis of about 0.001 to about 100 mg per kilogram (mg/kg) of body weight per day, or alternatively, about 0.01 to about 25 mg/kg of body weight per day, can be appropriate.

The compounds of Formula I can be administered orally, or alternatively, parenteral routes such as the subcutaneous, intramuscular, intravenous or intraperitoneal routes or any other suitable delivery system, such as intranasal or transdermal routes can also be employed.

As used herein, the phrase “taste modulating protein” refers to a TRPM5 protein. This protein is an ion channel that is a part of the taste-perception machinery. It has been shown to be essential for taste transduction. Perez et al., Nature Neuroscience 5:1169-1176 (2002); Zhang et al., Cell 112:293-301 (2003). Because this protein is a necessary part of the taste-perception machinery, modulation of its activity could modulate the sensation of particular tastes.

Taste is the ability to respond to dissolved molecules and ions called tastants. Humans detect taste with taste receptor cells, which are clustered in taste buds. (Kinnamon, S. C. TINS 11:491-496 (1988)). Tastants bind specific receptors on the cell membrane of a taste receptor cell, leading to a voltage change across the cell membrane. A change in voltage across the cell membrane depolarizes, or changes the electric potential of the cell. This leads to a signal being sent to a sensory neuron leading back to the brain.

TRPM5 is a member of the transient receptor potential (TRP) family of ion channels. Ion channels are transmembrane proteins that form pores in a cell membrane and allow ions to pass from one side to the other (reviewed in B. Hille (Ed), Ionic Channels of Excitable Membranes 2d ed., Sinauer, Sunderland, Mass. (1992)). Many channels have “gates” that open in response to a specific stimulus. As examples, voltage-gated channels respond to a change in the electric potential across the membrane, mechanically-gated channels respond to mechanical stimulation of the membrane, and ligand-gated channels respond to the binding of specific molecules. Various ligand-gated channels can open in response to extracellular factors, such as a neurotransmitters (transmitter-gated channels), or intracellular factors, such as ions (ion-gated channels), or nucleotides (nucleotide-gated channels). Still other ion channels are modulated by interactions with other proteins, such as G-proteins (G-protein coupled receptors or GPCRs).

Most ion channels mediate the permeation of one predominant ionic species. For example, sodium (Na⁺), potassium (K⁺), chloride (Cl⁻), and calcium (Ca²⁺) channels have been identified.

While not intending to be bound by theory, TRPM5 is believed to be activated by stimulation of a receptor pathway coupled to phospholipase C and by IP3-mediated Ca²⁺ release. The opening of this channel is dependent on a rise in Ca²⁺levels. Hofmann et al., Current Biol. 13:1153-1158 (2003). The activation of this channel leads to depolarization of a taste receptor cell, which in turn leads to transmitter release and excitation of primary gustatory nerve fibers. This protein is believed to mediate the permeation of monovalent cations.

Taste modulating protein includes naturally and recombinantly produced TRPM5 proteins; natural, synthetic, and recombinant biologically active polypeptide fragments of the protein; biologically active polypeptide variants of the protein or fragments thereof, including hybrid fusion proteins and dimers; biologically active polypeptide analogs of the protein or fragments or variants thereof, including cysteine substituted analogs. The taste modulating protein can be a human, or a non-human protein such as but not limited to a cow, horse, sheep, pig, chicken, turkey, quail, cat, dog, mouse, rat, rabbit, monkey, or guinea pig taste modulating protein. The taste modulating protein can be generated and/or isolated by any means known in the art. An example of the taste modulating protein and methods of producing the protein are disclosed in, for example, Liu and Liman, Proc. Nat'l Acad. Sci. USA 100: 15160-15165 (2003); D. Prawitt, et al., Proc. Nat'l Acad. Sci. USA 100:15166-15171 (2003); and Ulrich, N. D., et al., Cell Calcium 37:267-278 (2005); each of which is fully incorporated by reference herein.

An analog is a protein that can include one or more amino acid substitutions, deletions, or additions, either from natural mutations of human manipulation. Thus, by way of example, a taste modulating protein can include one or more amino acid substitutions, deletions or additions, either from natural mutations or human manipulation. As indicated, changes are preferably of a minor nature, such as conservative amino acid substitutions that do not significantly affect the folding or activity of the protein.

The variant taste modulating proteins which can be inhibited in accordance with the present invention comprise non-conservative modifications (e.g., substitutions). By “non-conservative” modification herein is meant a modification in which the wild-type residue and the mutant residue differ significantly in one or more physical properties, including hydrophobicity, charge, size, and shape. For example, modifications from a polar residue to a nonpolar residue or vice-versa, modifications from positively charged residues to negatively charged residues or vice versa, and modifications from large residues to small residues or vice versa are non-conservative modifications. For example, substitutions can be made which more significantly affect: the structure of the polypeptide backbone in the area of the alteration, for example the alpha-helical or beta-sheet structure; the charge or hydrophobicity of the molecule at the target site; or the bulk of the side chain. The substitutions which in general are expected to produce the greatest changes in the polypeptide's properties are those in which (a) a hydrophilic residue, e.g., seryl or threonyl, is substituted for (or by) a hydrophobic residue, e.g., leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine. In one embodiment, the variant taste modulating proteins used in accordance with the present invention have at least one non-conservative modification.

The present invention is also directed to a method of masking a taste, the method comprising administering to a subject in need of taste masking one or more compounds of Formula I, described above.

As used herein, the phrase “masking a taste” and grammatical variants thereof, such as “taste masking,” and “taste inhibiting” refers to interfering with the perception of a taste. By practicing the method of the present invention, the taste can be sensed to a lesser degree or not sensed at all.

The method of the present invention in its various embodiments can be used to mask one or more tastes selected from the group consisting of sweet, bitter, sour, salty, or umami. In some embodiments, the method of the present invention masks a bitter taste.

The method can be performed such that the taste to be masked by the compound of Formula I is masked by at least about 10%, 20%, 30%, 40%, 50%, 60%, 70%, 80%, 90%, or 95%, or at least about 10% to about 95%, or alternatively, by at least about 30% to about 75% (i.e., the sensing by a subject of the taste to be masked is reduced by at least about 10%, 20%, 30%, 40%, 50%, 60%, 70%, 80%, 90%, or 95%, or at least about 10% to about 95%, or alternatively, by at least about 30% to about 75%).

Any amount of the compound of Formula I that provides the desired degree of taste inhibiting can be used. For example, a compound of Formula I can be used at a concentration of about 0.1 μM to about 5,000 μM to inhibit a bitter taste. Alternatively, concentrations of about 1 μM, 100 μM, or 500 μM of a compound of Formula I can be used to inhibit a bitter taste.

In some embodiments, the taste masking effective amount of a compound of Formula I, or any of the specific subgroups, subclasses, or specific compounds described above, has a range of about 0.01 mg to about 5.0 grams per 100 mL. In some embodiments, the taste masking effective amount of a compound of Formula I, or any of the specific subgroups, subclasses, or specific compounds described above, has a range of about 0.5 mg to about 2 grams per 100 mL, about 0.1 grams to about 2 grams per 100 mL, or about 0.5 grams to about 2 grams per 100 mL. In some embodiments, a compound of Formula I, or any of the specific subgroups, subclasses, or specific compounds described above, is administered in an amount of about 1 gram per 100 mL.

In each of the methods of masking a taste described herein, a compound of Formula I, or any of the specific subgroups, subclasses, or specific compounds described above, is administered in an amount effective to mask the taste.

A taste to be masked can be an undesirable taste that is present in a pharmaceutical, veterinary, food, cosmetic or dental hygienic composition. Other compositions not explicitly listed herein, but which can contain components having undesirable tastes, are also within the scope of the present invention.

In some embodiments, a compound of Formula I can be present in the pharmaceutical, veterinary, food, cosmetic or dental hygienic composition having a taste to be masked, or alternatively, a compound of Formula I can be administered to a subject in need of taste masking by way of a pharmaceutical, veterinary, food, cosmetic or dental hygienic composition in addition to the pharmaceutical, veterinary, food, cosmetic or dental hygienic composition having a taste to be masked.

Thus, in some embodiments, the present invention is directed to a method of masking a taste of a pharmaceutical composition, comprising administering a compound of Formula I, or any of the specific subgroups, subclasses, or specific compounds described above, to a subject receiving the pharmaceutical composition. The compound of Formula I can be administered together with the pharmaceutical composition as a separate composition, for example, either concurrently or sequentially. The compound of Formula I can also be administered, or caused to be administered, prior to administering the pharmaceutical composition having the taste to be masked. Alternatively, the compound for Formula I can be administered as a component of the pharmaceutical composition having a taste to be masked.

In some embodiments, the method of the present invention comprises administering to a subject in need of taste masking a pharmaceutical composition comprising a biologically active agent other than a compound of Formula I. In some embodiments, a pharmaceutical composition comprising a biologically active agent other than a compound of Formula I can have an unpleasant or undesirable taste. Such an undesirable taste can be a bitter taste. In some embodiments, a bitter taste can be caused by one or more biologically active agents other than a compound of Formula I. Thus, the method of the present invention for masking an undesirable taste comprises administering a compound of Formula I to mask the taste of a biologically active agent that is other than a compound of Formula I.

By way of additional examples, the method of masking a taste of a pharmaceutical composition can comprise masking a taste produced by one or more biologically active agents selected from the group consisting of analgesics, anesthetics, anorexiants, appetite depressants, antacids, antiasthmatics, antidiuretics, antipyretics, antihistamines, anticholinergics, antidiarrheals, antitussives, antinauseants, antiarrhythmics, antimicrobials, antibacterials, antifungals, antivirals, anti-inflammatory agents, agents active against flatulence, antimigraine agents, beta-receptor blockers, bronchodilators, psychopharmacological agents, spasmolytics, sedatives, antihyperkinetics, tranquilizers, decongestants, demulcents, agents for alcohol withdrawal, antitussives, fluorine supplements, laxatives, local antibiotics, corticosteroid supplements, agents against goiter formation, antiepileptics, agents against dehydration, antiseptics, NSAIDs, H₂-receptor antagonists, nutritional supplements, gastrointestinal active agents, alkaloids, supplements for trace elements, ion-exchange resins, cholesterol-depressant agents, lipid-lowering agents, expectorants, and combinations thereof. Further specific examples of pharmaceutical compositions and biologically active agents having tastes to be masked in accordance with the method of the invention are described below.

In some embodiments, the one or more compounds of Formula I are administered to a subject in need of taste masking, or are present in a pharmaceutical composition having an undesirable taste, in an amount sufficient to mask or inhibit an undesirable taste produced by a biologically active agent by at least about 10%, 20%, 30%, 40%, 50%, 60%, 70%, 80%, 90%, or 95%, or by at least about 10% to about 95%, or alternatively, by at least about 30% to about 75%.

The compound of Formula I can be administered to a subject in need of taste masking a biologically active agent in a ratio of about 1000:1 to about 1:1000, or alternatively, about 100:1 to about 1:100, relative to the amount of the biologically active agent administered.

For example, the present invention is directed to a method of inhibiting a bitter taste of a pharmaceutical composition, comprising administering to a subject in need of such method a pharmaceutical composition and a compound of Formula I, wherein the pharmaceutical composition comprises a pharmaceutically active agent and optionally one or more excipients, and wherein the compound of Formula I is administered as either a component of the pharmaceutical composition or as a separate dosage form, and wherein molar ratio of the compound of Formula I to the pharmaceutically active agent about 1000:1 to about 1:1000, or alternatively administered in a molar ratio of about 500:1, about 200:1, about 10:1, about 1:1, about 1:10, about 1:200, or about 1:500. As will be appreciated, the various ranges and amounts of the compound of Formula I can be used, with modifications if preferred, in each of the embodiments described herein.

Additionally, the method of taste masking a pharmaceutical composition can comprise masking a taste produced by a counterterrorism pharmaceutical agent. A counterterrorism pharmaceutical agent includes those pharmaceutical agents that are useful in counteracting agents that could be used in a terrorist attack. A terrorist attack could result in exposure of human and/or animal subjects to chemical, nuclear, and/or biological weapons. In case of such exposure, counterterrorism pharmaceutical agents can be used to counteract the effects of chemical, nuclear, and/or biological weapons. Possible active agents released by such chemical, nuclear, and/or biological weapons include, but are not limited to, chemical agents such as ricin, sarin, tabun, soman, methylphosphonothoic acid, sulfur mustard, and nitrogen mustard; nuclear and radioactive agents such as x-rays, α and β particles, and γ radiation; and biological agents such as anthrax, SARS virus, smallpox virus, and avian influenza virus. Biological active agents that counteract such agents are useful as a counterterrorism pharmaceutical. Thus, counterterrorism pharmaceuticals include, but are not limited to, antibiotics such as ciprofloxacin and doxycycline; potassium iodide; and antiviral agents.

Thus, in some embodiments of the present invention, an undesirable taste of a counterterrorism pharmaceutical, such as an antibiotic such as ciprofloxacin and doxycycline; potassium iodide; or an antiviral agent, is masked by a compound of Formula I by at least about 10%, 20%, 30%, 40%, 50%, 60%, 70%, 80%, 90%, or 95%, or by at least about 10% to about 95%, or alternatively, by at least about 30% to about 75%.

The compound of Formula I can be administered to a subject in need of taste masking a counterterrorism agent in a ratio of about 1000:1 to about 1:1000, or alternatively, about 100:1 to about 1:100, relative to the amount of the counterterrorism agent administered.

In some embodiments, a compound of Formula I, or any of the specific subgroups, subclasses, or specific compounds described above, is useful for masking an undesirable taste of a nutriceutical composition. Examples of nutriceutical compositions having an undesirable taste include, but are not limited to, enteral nutrition compositions for treatment of nutritional deficit, trauma, surgery, Crohn's disease, renal disease, hypertension, obesity and the like, enhancement of athletic performance, muscle growth, or general well being, or treatment of inborn errors of metabolism such as phenylketonuria. In particular, such nutriceutical formulations can contain one or more amino acids which have a bitter or metallic taste or aftertaste. Such amino acids include, but are not limited to, an essential amino acid selected from the group consisting of L-isomers of leucine, isoleucine, histidine, lysine, methionine, phenylalanine, threonine, tryptophan, tyrosine, valine, and combinations thereof. Further specific examples of nutriceutical compositions in accordance with the method of the invention are described below.

In some embodiments, a compound of Formula I, or any of the specific subgroups, subclasses, or specific compounds described above, is used to inhibit a bitter taste associated with one or more the following: bitter pharmaceutical alkaloids such as acetaminophen, ampicillin, chlorpheniramine, clarithromycin, doxylamine, guaifenesin, ibuprofen, pseudoephidrine hydrochloride, and ranitidine; bitter pharmaceutical metallic salts such as zinc-containing bioadhesives (denture adhesive); bitter vitamins; bitter components of foods such as creatine, limonine, naringin, quinizolate; and bitter components of beverages such as caffeine, and humulone. In one embodiment, the concentration of the compound of Formula I used is in the range of 0.01 mM to 20 mM. The amount can vary depending on the amount of bitter compound used and its bitterness.

The method of the present invention also comprises administering a nutraceutical composition comprising a nutraceutical agent, optionally one or more excipients, and one or more compounds of Formula I, wherein the one or more compounds of Formula I are present in an amount sufficient to mask an undesired taste produced by the nutraceutical agent, by at least about 10%, 20%, 30%, 40%, 50%, 60%, 70%, 80%, 90%, or 95%, or at least about 10% to about 95%, or alternatively, by at least about 30% to about 75%.

A compound of Formula I can also be incorporated into a medical and/or dental composition. Certain medical and/or dental compositions used in diagnostic procedures have an unpleasant taste, such as contrast materials and local anesthetics. Thus, it is within the scope of the present invention to administer one or more compounds of Formula I to a subject undergoing a medical and/or dental procedure (i.e., an imaging or surgical procedure) to improve the comfort of the subjects by masking an undesirable taste of a medical and/or dental composition used during the procedure. In addition, the taste masking compounds of the present invention can be incorporated into pharmaceutical compositions, including tablets and liquids, to improve their flavor and improve patient compliance, particularly where the patient is a child or an animal.

In some embodiments, the present invention is directed to a method of masking the undesirable taste of a veterinary composition, such as veterinary medicines, veterinary food compositions, veterinary supplements, and the like, that are administered to domesticated animals. In a preferred embodiment, a compound of Formula I, or any of the specific subgroups, subclasses, or specific compounds described above, is used to mask a taste of a veterinary composition administered to a cat or dog.

In some embodiments, a taste to be masked can be an undesirable taste of a food composition. Examples of food compositions having an undesirable taste include, but are not limited to, citrus fruits such as grapefruit, orange, and lemon; vegetables such as tomato, pimento, celery, melon, carrot, potato and asparagus; seasoning or flavoring materials, such as soy sauce and red pepper; soybean compositions; fish; meats and processed meats; dairy such as cheese, yogurt, cream, and milk; breads; cakes; and confectioneries such as candies, chewing gum and chocolate. Other examples of food compositions envisioned in accordance with the present invention are described below and throughout the specification.

A food composition can also include beverages and drinks. Examples of drinks having an undesirable or unwanted taste include, but are not limited to, juices of citrus fruits and vegetables, soybean, milk, coffee, cocoa, black tea, green tea, fermented tea, semi-fermented tea, refreshing drinks, beverages and milk.

Thus, in some embodiments, the method of the present invention comprises administering to a subject in need of taste masking a food composition comprising one or more food ingredients and one or more compounds of Formula I, wherein the one or more compounds of Formula I are present in the food composition in an amount sufficient to mask a bitter taste produced by an ingredient in the food composition by at least about 10%, 20%, 30%, 40%, 50%, 60%, 70%, 80%, 90%, or 95%, or by at least about 10% to about 95%, or alternatively, by at least about 30% to about 75%.

In some embodiments, a compound of Formula I, or any of the specific subgroups, subclasses, or specific compounds described above, is used to inhibit a taste of a cosmetic composition. For example, but not by way of limitation, a compound of Formula I can be incorporated into face cream, lipstick, lip gloss, and the like. Additionally, a compound of Formula I, or any of the specific subgroups, subclasses, or specific compounds described above, can be used to inhibit an unpleasant taste of a lip balm, such as CHAPSTICK® (Wyeth Corporation, Madison, N.J.) or BURT'S BEESWAX® (Burt's Bees, Inc. Durham, N.C.).

In addition, a compound of Formula I, or any of the specific subgroups, subclasses, or specific compounds described above, can be incorporated into compositions that are not traditional foods, pharmaceuticals, or cosmetics, but which can contact taste membranes. Examples include, but are not limited to, soaps, shampoos, toothpaste, denture adhesive, and glue on the surfaces of stamps and envelopes. Thus, the scope of the present invention also is directed to a method of masking an undesirable taste produced by one or more components of a composition that is not a traditional food, pharmaceutical, or cosmetic, but which can contact taste membranes, the method comprising adding a compound of Formula I to the composition. The present invention is also directed to a process for preparing an improved composition that is not a traditional food, pharmaceutical, or cosmetic, but which can contact taste membranes, wherein the improvement comprises adding a compound of Formula I to the composition.

In some embodiments, in the taste inhibiting methods described herein, a compound of Formula I, or any of the specific subgroups, subclasses, or specific compounds described above, is administered in an amount that is sufficient, in combination with the administration of one or more additional taste inhibiting agents, to inhibit the taste. For example, in a method of inhibiting the bitter taste of a liquid pharmaceutical composition, the composition comprises a compound of Formula I and another taste inhibiting agent, wherein the amount of the compound of Formula I is about 25% to about 75% of the amount required to inhibit the bitter taste in the absence of the other taste inhibiting agent.

The present invention is also directed to a method of inhibiting the depolarization of a taste receptor cell, comprising contacting the taste receptor cell with one or more compounds of Formula I, or any of the specific subgroups, subclasses, or specific compounds described above.

Not being bound by any particular theory, a compound of Formula I can inhibit the depolarization of a taste receptor cell by a mechanism other than, or in addition to, the mechanism of inhibiting a taste receptor protein. The method of the present invention comprises administering a compound of Formula I, or any of the specific subgroups, subclasses, or specific compounds described above, to a subject in an amount sufficient to inhibit the depolarization of a taste receptor cell. Furthermore, when administered orally, the compound of Formula I can be administered with one or more pharmaceutically acceptable carriers. Additionally, the compound of Formula I, and/or the carrier administered with it, can be dispersed or diluted by saliva.

In some embodiments, the method of the present invention comprises contacting a taste receptor cell with a compound of Formula I, or any of the specific subgroups, subclasses, or specific compounds described above, wherein the taste receptor cell can detect a sweet, bitter, sour, salty, or umami taste. In some embodiments, the taste receptor cell is a human taste receptor cell, or alternatively, an animal taste receptor cell.

By way of example, the present invention is directed to a method of inhibiting the depolarization of a taste receptor cell, comprising contacting the taste receptor cell with a compound of Formula I, or any of the specific subclasses and specific compounds listed above, and inhibiting the depolarization of the taste receptor cell by at least about 10%, 20%, 30%, 40%, 50%, 60%, 70%, 80%, 90%, or 95%, or by at least about 10% to about 95%, or alternatively, by at least about 30% to about 75%.

In some embodiments, the present invention is directed to a method of inhibiting the depolarization of a taste receptor cell, comprising contacting a protein within the taste receptor cell with a compound of Formula I, or any of the specific subclasses and specific compounds listed above, wherein the protein is a naturally occurring taste-modulating protein in a taste receptor cell.

In some embodiments the taste receptor cell is a human taste receptor cell.

Any amount of the compound of Formula I that provides the desired degree of inhibition can be used. For example, a compound of Formula I can be used at a concentration of about 0.1 μM to about 1,000 μM to inhibit the depolarization of a taste receptor cell. Alternatively, a concentration of about 1 μM, 50 μM, or 100 μM of a compound of Formula I can be used to inhibit the depolarization of a taste receptor cell.

In some embodiments, a single dose or two to four divided daily doses, provided on a basis of about 0.001 to 100 mg per kilogram of body weight per day, or alternatively, about 0.01 to about 25 mg/kg of body weight per day is appropriate to inhibit the depolarization of a human or animal taste receptor cell. When inhibiting the depolarization of a taste receptor cell in vivo, the compound of Formula I can be administered orally.

In some embodiments, the taste receptor cell in which depolarization is inhibited can sense a taste produced by a biologically active agent. Biologically active agents whose taste can be sensed by a taste receptor cell whose depolarization can be inhibited by the method of the present invention include analgesics, anesthetics, anorexiants, appetite depressants, antacids, antiasthmatics, antidiuretics, antipyretics, antihistamines, anticholinergics, antidiarrheals, antitussives, antinauseants, antiarrhythmics, antimicrobials, antibacterials, antifungals, antivirals, anti-inflammatory agents, agents active against flatulence, antimigraine agents, beta-receptor blockers, bronchodilators, psychopharmacological agents, spasmolytics, sedatives, antihyperkinetics, tranquilizers, decongestants, demulcents, agents for alcohol withdrawal, antitussives, fluorine supplements, laxatives, local antibiotics, corticosteroid supplements, agents against goiter formation, antiepileptics, agents against dehydration, antiseptics, H₂-receptor antagonists, nutritional supplements, NSAIDs, gastrointestinal active agents, alkaloids, supplements for trace elements, ion-exchange resins, cholesterol-depressant agents, lipid-lowering agents, expectorants, and combinations thereof.

The present invention is also directed to a method of decreasing the palatability of food in a subject, the method comprising administering to a subject in need of such treatment one or more compounds of Formula I, or any of the specific subgroups, subclasses, or specific compounds described above, in an amount sufficient to decrease the caloric intake by the subject. In some embodiments, the present invention is also directed to diminishing the caloric intake in a subject, the method comprising administering to a subject one or more compounds of Formula I, or any of the specific subgroups, subclasses, or specific compounds described above, in an amount sufficient to decrease the caloric intake in the subject.

Taste modulating protein knockout mice have been shown to have diminished taste preference for sucrose, artificial sweeteners, and umami flavors and diminished taste aversion to bitter solutions. See Zhang et al., Cell 112:293-301 (2003). Thus, in the present invention, a compound of Formula I, or any of the specific subgroups, subclasses, or specific compounds described above, can be administered to a subject to reduce the palatability of food to the subject. Not being bound by any particular theory, a reduced palatability of food in a subject can lead to a lower intake of food by the subject, thereby reducing the caloric intake by a subject. Thus, in some embodiments, by administering a compound of Formula I, or any of the specific subgroups, subclasses, or specific compounds described above, to a subject, the subject will consume a decreased amount of food compared to the subject's food intake when not being administered a compound of Formula I, or any of the specific subgroups, subclasses, or specific compounds described above. And in some embodiments, by administering a compound of Formula I, or any of the specific subgroups, subclasses, or specific compounds described above, to a subject, the subject will consume a decreased amount of calories compared to the subject's caloric intake when not being administered a compound of Formula I, or any of the specific subgroups, subclasses, or specific compounds described above. Additionally, administering a compound of Formula I, or any of the specific subgroups, subclasses, or specific compounds described above, to a subject can be a method of treating food-related psychological disorders, a method of dieting, or a method of facilitating weight loss in a subject in need thereof.

In each of the embodiments described above, the subject of the method, unless otherwise limited, can be any animal which is in need of the particular treatment or effect of the method. Such animals include but are not limited to a cow, horse, sheep, pig, chicken, turkey, quail, cat, dog, mouse, rat, rabbit, monkey, guinea pig, or any other animal having a taste modulating protein. In some embodiments, the animal is a livestock animal, a domesticated animal, or an animal kept as a pet. In some embodiments, the subject of the claimed method is a human.

In some embodiments, the palatability of food can be reduced in a subject suffering from compulsive overeating disorder, bulimia, binge eating disorder, obesity, over-eating, or eating without care for stopping.

In some embodiments, the caloric intake by a subject can be reduced in a subject suffering from type-II diabetes, or other diabetes related disorders, or alternatively having a body mass index (BMI) greater than about 30. In some embodiments a subject in need of reduced caloric intake has a BMI of about 30 to about 50.

In some embodiments, the method of the present invention reduces the caloric intake in a subject in need of such treatment by at least about 10%. In some embodiments, the present invention is directed to a method of inhibiting the caloric intake by a subject, comprising administering the subject in need of such treatment a compound of Formula I, or any of the specific subclasses or specific compounds listed above, and decreasing the caloric intake by at least about 10%, 20%, 30%, 40%, 50%, 60%, 70%, 80%, 90%, or 95%, or by at least about 10% to about 95%, or alternatively, by at least about 30% to about 75%.

In some embodiments, the method of reducing the palatability of food of reducing the caloric intake by a subject comprises administering a compound of Formula I as a pharmaceutical or food composition.

Furthermore, in each of the embodiments of the methods described herein, a compound of Formula I can be administered as a pharmaceutical, veterinary, food, cosmetic, or dental hygienic composition. And in any of the method described herein, a compound of Formula I can be present in a pharmaceutical, veterinary, food, cosmetic, or dental hygienic composition in a concentration of about 0.01% to about 50%, by weight, of the pharmaceutical, food, cosmetic, or dental hygienic composition. Alternatively, a compound of Formula I can be present in pharmaceutical, veterinary, food, cosmetic, or dental hygienic composition for use by a method of the present invention in a concentration of about 0.5% to about 20%, or alternatively, about 1% to about 10%, by weight, of the pharmaceutical, food, cosmetic, or dental hygienic composition.

Furthermore, in any of the method described herein, a compound of Formula I can be present in a pharmaceutical, veterinary, food, cosmetic, or dental hygienic composition in an amount of about 0.01 mg to about 100 mg. Alternatively, a compound of Formula I can be present in an amount of about 0.1 mg to about 50 mg, or alternatively, about 0.5 mg to about 20 mg.

Furthermore, in each of the embodiments of the methods described herein, a compound of Formula I can be used in varying ratios to the agent that is believed to cause the undesirable taste, such as a bitter taste. For example, a compound of Formula I can be administered in a molar ratio of about 1000:1 to about 1:1000, or alternatively administered in a molar ratio of about 500:1, about 200:1, about 10:1, about 1:1, about 1:10, about 1:200, or about 1:500, relative to the agent that is believed to cause the undesirable taste.

Compositions

The present invention is also directed to various, useful compositions comprising a compound of Formula I or a pharmaceutically acceptable salt thereof.

In one aspect, the present invention is directed to a pharmaceutical composition comprising a compound of Formula I, as defined above, including any of the specific embodiments, subclasses, or species described above, and one or more pharmaceutically acceptable carriers. Preferred compositions of the present invention are pharmaceutical compositions comprising a compound selected from one or more embodiments listed above, and one or more pharmaceutically acceptable excipients. Pharmaceutical compositions that comprise one or more compounds of Formula I, or any of the specific subgroups, subclasses, or specific compounds described above, can be used to formulate pharmaceutical drugs containing one or more active agents that exert a biological effect other than taste masking and/or inhibition of a taste modulating protein.

Such active agents are well known in the art. See, e.g., The Physician's Desk Reference. Such compositions can be prepared using procedures known in the art, for example, as described in Remington: The Science and Practice of Pharmacy, 21st Ed. Lippincott Williams & Wilkins, Baltimore, Md. (2003). In some embodiments, a biologically active agent having an activity other than taste masking for use with a pharmaceutical composition of the present invention can include, but is not limited to, a bronchodilator, anorexiant, antihistamine, nutritional supplement, laxative, analgesic, anesthetic, antacid, H₂-receptor antagonist, anticholinergic, antidiarrheal, demulcent, antitussive, NSAID, antinauseant, antimicrobial, antibacterial, antifungal, antiviral, expectorant, anti-inflammatory agent, antipyretic, and combinations thereof. The pharmaceutical composition of the present invention can comprise one or more compounds of Formula I, as described above, or any of the specific subgroups, subclasses, or specific compounds described above; an active agent that has a bitter taste; and optionally one or more pharmaceutically acceptable carriers.

In some embodiments, the biologically active agent is selected from the group consisting of antipyretics and analgesics, e.g., ibuprofen, acetaminophen, or aspirin; laxatives, e.g., phenolphthalein dioctyl sodium sulfosuccinate; appetite depressants, e.g., amphetamines, phenylpropanolamine, phenylpropanolamine hydrochloride, or caffeine; antacids, e.g., calcium carbonate; antiasthmatics, e.g., theophylline; antidiuretics, e.g., diphenoxylate hydrochloride; agents active against flatulence, e.g., simethecone; migraine agents, e.g., ergotaminetartrate; psychopharmacological agents, e.g., haloperidol; spasmolytics or sedatives, e.g., phenobarbitol; antihyperkinetics, e.g., methyldopa or methylphenidate; tranquilizers, e.g., benzodiazepines, hydroxinmeprobramates or phenothiazines; antihistaminics, e.g., astemizol, chloropheniramine maleate, pyridamine maleate, doxlamine succinate, bromopheniramine maleate, phenyltoloxamine citrate, chlorocyclizine hydrochloride, pheniramine maleate, and phenindamine tartrate; decongestants, e.g., phenylpropanolamine hydrochloride, phenylephrine hydrochloride, pseudoephidrine hydrochloride, pseudoephidrine sulfate, phenylpropanolamine bitartrate, and ephedrine; beta-receptor blockers, e.g., propanolol; agents for alcohol withdrawal, e.g., disulfiram; antitussives, e.g., benzocaine, dextromethorphan, dextromethorphan hydrobromide, noscapine, carbetapentane citrate, and chlophedianol hydrochloride; fluorine supplements, e.g., sodium fluoride; local antibiotics, e.g., tetracycline or cleocine; corticosteroid supplements, e.g., prednisone or prednisolone; agents against goiter formation, e.g., colchicine or allopurinol; antiepileptics, e.g., phenytoine sodium; agents against dehydration, e.g., electrolyte supplements; antiseptics, e.g., cetylpyridinium chloride; NSAIDs, e.g., acetaminophen, ibuprofen, naproxen, or salts thereof; gastrointestinal active agents, e.g., loperamide and famotidine; various alkaloids, e.g., codeine phosphate, codeine sulfate, or morphine; supplements for trace elements, e.g., sodium chloride, zinc chloride, calcium carbonate, magnesium oxide, and other alkali metal salts and alkali earth metal salts; vitamins; ion-exchange resins, e.g., cholestyramine; cholesterol-depressant and lipid-lowering substances; antiarrhythmics, e.g., N-acetylprocainamide; and expectorants, e.g., guaifenesin.

Biologically active agents that have a particularly unpleasant taste include antibacterial agents such as ciprofloxacin, ofloxacin, and pefloxacin; antiepileptics such as zonisamide; macrolide antibiotics such as erythromycin; beta-lactam antibiotics such as penicillins and cephalosporins; psychotropic active substances such as chlorpromazine; active substances such as sulpyrine; and agents active against ulcers, such as cimetidine.

In some embodiments, the pharmaceutical composition of the present invention comprises one or more compounds of Formula I, or any of the specific subgroups, subclasses, or specific compounds described above, and at least one amino acid selected from the group consisting of glycine, L-alanine, L-arginine, L-aspartic acid, L-cystine, L-glutamic acid, L-glutamine, L-histidine, L-isoleucine, L-leucine, L-lysine, L-methionine, L-ornithine, L-phenylalanine, L-proline, L-serine, L-threonine, L-tryptophan, L-tyrosine, L-valine, creatine, and mixtures thereof.

In some embodiments, the pharmaceutical composition of the present invention comprises one or more compounds of Formula I, or any of the specific subgroups, subclasses, or specific compounds described above; a biologically active agent that exhibits an activity other than taste inhibition; and at least one amino acid, such as one selected from the group consisting of glycine, L-alanine, L-arginine, L-aspartic acid, L-cystine, L-glutamic acid, L-glutamine, L-histidine, L-isoleucine, L-leucine, L-lysine, L-methionine, L-ornithine, L-phenylalanine, L-proline, L-serine, L-threonine, L-tryptophan, L-tyrosine, L-valine, creatine, and mixtures thereof.

The pharmaceutical compositions of the present invention can be in any form suitable to achieve their intended purpose. Preferably, however, the composition is one which can be administered buccally or orally. Alternatively, the pharmaceutical composition can be an oral or nasal spray.

The pharmaceutical compositions of the invention can be in any form suitable for administration to any subject that can experience the beneficial effects of one or more compounds of Formula I, or any of the specific subgroups, subclasses, or specific compounds described above. In some embodiments, the subject is a human, although the invention is not intended to be so limited. Other suitable subjects include the following animals: cow, horse, sheep, pig, chicken, turkey, quail, cat, dog, mouse, rat, rabbit, monkey, and guinea pig. A veterinary composition, as used herein, refers to a pharmaceutical composition that suitable for non-human animals. Such veterinary compositions are known in the art.

The pharmaceutical compositions of the present invention can be manufactured using known methods, for example, by means of conventional mixing, granulating, dragée-making, dissolving, or lyophilizing processes. Thus, pharmaceutical preparations for oral use can be obtained by combining the active compounds with solid excipients, optionally grinding the resulting mixture, and processing the mixture of granules, after adding suitable auxiliaries, if desired or necessary, to obtain tablets or dragée cores.

Pharmaceutical excipients are well known in the art. Suitable excipients include fillers such as saccharides, for example, lactose or sucrose, mannitol or sorbitol, cellulose preparations and/or calcium phosphates, for example, tricalcium phosphate or calcium hydrogen phosphate, as well as binders, such as, starch paste, using, for example, maize starch, wheat starch, rice starch, potato starch, gelatin, tragacanth, methylcellulose, hydroxypropylmethylcellulose, sodium carboxymethylcellulose, and/or polyvinyl pyrrolidone. If desired, disintegrating agents can be added, such as, the above-mentioned starches and also carboxymethyl-starch, cross-linked polyvinyl pyrrolidone, agar, or alginic acid or a salt thereof, such as, sodium alginate. Suitable excipients also include flow-regulating agents and lubricants, for example, silica, talc, stearic acid or salts thereof, such as, magnesium stearate or calcium stearate, and/or polyethylene glycol. Dragée cores are provided with suitable coatings that, if desired, are resistant to gastric juices. For this purpose, concentrated saccharide solutions can be used, which can optionally contain gum arabic, talc, polyvinyl pyrrolidone (i.e., povidone), polyethylene glycol, and/or titanium dioxide, lacquer solutions and suitable organic solvents or solvent mixtures. To produce coatings resistant to gastric juices, solutions of suitable cellulose preparations, such as, acetylcellulose phthalate or hydroxypropylmethyl-cellulose phthalate, can be used. Dye stuffs or pigments can be added to the tablets or dragée coatings, for example, for identification or in order to characterize combinations of active compound doses.

Liquid dosage forms for oral administration include pharmaceutically acceptable emulsions, solutions, suspensions, syrups, and elixirs. In addition to the active compounds, the liquid dosage forms can contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Suspensions, in addition to the active compounds, can contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

In some embodiments, the invention is directed to a chewable tablet comprising one or more compounds of Formula I and one or more biologically active agents. Chewable tablets are known in the art. See, e.g., U.S. Pat. Nos. 4,684,534 and 6,060,078, each of which is incorporated by reference in its entirety. Any kind of medicament can be contained in the chewable tablet, preferably a medicament of bitter taste, natural plant extracts or other organic compounds. More preferably, vitamins such as vitamin A, vitamin B, vitamin B₁, vitamin B₂, vitamin B₆, vitamin C, vitamin E and vitamin K; natural plant extracts such as Sohgunjung-tang extracts, Sipchundaebo-tang extracts and Eleutherococcus senticosus extracts; organic compounds such as dimenhydrinate, meclazine, acetaminophen, aspirin, phenylpropanolamine, and cetylpyridinium chloride; or gastrointestinal agents such as dried aluminum hydroxide gel, domperidone, soluble azulene, L-glutamine and hydrotalcite can be contained in the core.

In some embodiments, the present invention is directed to an orally disintegrating composition wherein the orally disintegrating composition further comprises one or more compounds of Formula I, or any of the specific subgroups, subclasses, or specific compounds described above. Orally disintegrating dosage forms are known in the art. See, e.g., U.S. Pat. Nos. 6,368,625 and 6,316,029, each of which is hereby incorporated by reference in its entirety.

In some embodiments, the present invention is further directed to a nasal composition further comprising one or more compounds of Formula I, or any of the specific subgroups, subclasses, or specific compounds described above. Nasal sprays are known in the art. See, e.g., U.S. Pat. No. 6,187,332. Addition of one or more compounds of Formula I to a nasal spray can reduce the experience of an unpleasant taste associated with the composition of the nasal spray. By way of a nonlimiting example, a nasal spray composition of the present invention comprises water (such as approximately 95% to about 98% by weight), a citrate (such as, for example, about 0.02 M to about 0.06 M citrate anion), a compound of Formula I, and optionally phosphate (such as about 0.03 M to about 0.09 M phosphate).

In some embodiments, the present invention is directed to a solid dosage form comprising a water and/or saliva activated effervescent granule, such as one having a controllable rate of effervescence, and a compound of Formula I, or any of the specific subgroups, subclasses, or specific compounds described above. The effervescent composition can further comprise a pharmaceutically active compound. Effervescent pharmaceutical compositions are known in the art. See, e.g., U.S. Pat. No. 6,649,186, which is incorporated by reference in its entirety. The effervescent composition can be used in pharmaceutical, veterinary, horticultural, household, food, culinary, pesticidal, agricultural, cosmetic, herbicidal, industrial, cleansing, confectionery and flavoring applications. Formulations incorporating the effervescent composition comprising a compound of Formula I can further include one or more additional adjuvants and/or active ingredients which can be chosen from those known in the art including flavors, diluents, colors, binders, filler, surfactant, disintegrant, stabilizer, compaction vehicles, and non-effervescent disintegrants.

In some embodiments, the present invention is directed to a film-shaped or wafer-shaped pharmaceutical composition that comprises a compound of Formula I, or any of the specific subgroups, subclasses, or specific compounds described above, and is capable of disintegrating. Such a film-shaped or wafer-shaped pharmaceutical composition can be configured, for example, as quickly disintegrating administration forms, e.g., administration forms disintegrating within a period of 1 second up to about 2 minutes, or as slowly disintegrating administration forms, e.g., administration forms disintegrating within a period of about 2 to about 15 minutes.

The indicated disintegration times can be set to the above-mentioned ranges by using, for example, matrix-forming polymers which have different disintegrating, or solubility, characteristics. Thus, by mixing the corresponding polymer components, the disintegration time can be adjusted. In addition, disintegrants are known which “draw” water into the matrix and cause the matrix to burst open from within. As a consequence, certain embodiments of the invention include such disintegrants for the purpose of adjusting the disintegration time.

Suitable polymers for use in the film-shaped or wafer-shaped pharmaceutical composition include cellulose derivatives, polyvinyl alcohol (e.g., MOWIOL™, Hoechst Aktiengesellschaft, Frankfurt, Germany), polyacrylates, polyvinyl pyrrolidone, cellulose ethers, such as ethyl cellulose, as well as polyvinyl alcohol, polyurethane, polymethacrylates, polymethylmethacrylates and derivatives and copolymerisates of the aforementioned polymers.

In some embodiments, the total thickness of the film-shaped or wafer-shaped pharmaceutical composition of the invention is preferably 5 μm to about 10 mm, or 30 μm to about 2 mm, or alternatively, about 0.1 mm to about 1 mm. The pharmaceutical preparations can be of round, oval, elliptic, triangular, quadrangular or polygonal shape, but other suitable shapes are within the scope of the present invention.

In some embodiments, the present invention is directed to a composition comprising a medicament or agent contained in a coating that surrounds a gum base formulation and further comprising a taste-inhibiting amount of a compound of Formula I, or any of the specific subgroups, subclasses, or specific compounds described above. Preferably, the coating comprises at least 50%, by weight, of the entire composition. As the center is chewed, the medicament or agent is released into the saliva. For example, U.S. Pat. No. 6,773,716, which is incorporated herein by reference in its entirety, discloses a suitable medicament or agent contained in a coating that surrounds a gum base formulation. One or more compounds of Formula I, or any of the specific subgroups, subclasses, or specific compounds described above, can be used in preparing the coating. Optionally, the composition can further comprise high-intensity sweeteners and appropriate flavors. It has been found that for certain medicaments or agents that have an astringent or bitter taste, adding an inhibiting agent to the formulation can provide a much more palatable formulation. In this regard, even though the medicament in, for example, its powder form can be bitter or have an offensive taste, the matrix used as the coating of the present invention, including the inhibiting agent, will afford a composition having acceptable medicinal properties. The compound of Formula I, or any of the specific subgroups, subclasses, or specific compounds described above, can be present in varying amounts, such as about 1%, 30% 50%, 75%, 90%, or 99%, or can be present in about 30% to about 99%, or alternatively, can be present in about 1% to about 30%, by weight, of the composition.

The present invention is also directed to a process of preparing an improved pharmaceutical composition, wherein the improvement comprises adding a compound of Formula I, or any of the specific subgroups, subclasses, or specific compounds described above, to the pharmaceutical composition.

In some embodiments, the invention is directed to a pharmaceutical composition suitable for aerosol administration, comprising a compound of Formula I, or any of the specific subgroups, subclasses, or specific compounds described above, and a suitable carrier. The aerosol composition can further comprise a biologically active agent in addition to a compound of Formula I. Aerosol compositions are known in the art. See, e.g., U.S. Pat. No. 5,011,678, which is hereby incorporated by reference in its entirety. As a nonlimiting example, an aerosol composition of the present invention can comprise a medically effective amount of a pharmaceutically active substance, one or more compounds of Formula I, or any of the specific subgroups, subclasses, or specific compounds described above, and a biocompatible propellant, such as a (hydro/fluoro)carbon propellant.

In some embodiments, a pharmaceutical, veterinary, food, cosmetic, or dental hygienic composition of the present invention comprises about 0.01 mg to about 100 mg, or alternatively, about 0.01 mg to about 10 mg, of a compound of Formula I.

In some embodiments, a pharmaceutical, veterinary, food, cosmetic, or dental hygienic composition of the present invention comprises a compound of Formula I in an amount sufficient to inhibit a taste modulating protein. By way of example, the pharmaceutical, veterinary, food, cosmetic, or dental hygienic composition of the present invention can comprise a compound of Formula I in an amount sufficient to inhibit the taste modulating protein by at least about 10%, 20%, 30%, 40%, 50%, 60%, 70%, 80%, 90%, or 95%, or by at least about 10% to about 95%, or alternatively, by at least about 30% to about 75%.

In some embodiments, the present invention is directed to a nutriceutical composition comprising one or more nutriceuticals, one or more compounds of Formula I, or any of the specific subgroups, subclasses, or specific compounds described above, and optionally one or more carriers. Examples of nutriceutical compositions having an undesirable taste include, but are not necessarily limited to, enteral nutrition compositions for treatment of nutritional deficit, trauma, surgery, Crohn's disease, renal disease, hypertension, obesity and the like, to promote athletic performance, muscle enhancement or general well being or inborn errors of metabolism such as phenylketonuria. In particular, such nutriceutical formulations can contain one or more amino acids which have a bitter or metallic taste or aftertaste. Such amino acids include, but are not limited to, an essential amino acids selected from the group consisting of L-isomers of leucine, isoleucine, histidine, lysine, methionine, phenylalanine, threonine, tryptophan, tyrosine, and valine. Additionally, the invention is directed to a process of preparing an improved nutriceutical composition, wherein the improvement comprises adding one or more compounds of Formula I, or any of the specific subgroups, subclasses, or specific compounds described above, to a nutriceutical composition. In certain embodiments, the one or more compounds of Formula I, or any of the specific subgroups, subclasses, or specific compounds described above, are added to a nutriceutical composition in an amount of about 1% to about 50%, or about 5%, 10%, or 15%, by weight.

The present invention is also directed to a dental hygienic composition comprising one or more compounds of Formula I, or any of the specific subgroups, subclasses, or specific compounds described above. Dental hygienic compositions are known in the art and include but are not necessarily limited to toothpaste, mouthwash, plaque rinse, dental floss, dental pain relievers (such as ANBESOL™, Wyeth Corporation, Madison, N.J.), and the like. For example, the invention includes a dental bleaching composition which comprises one or more compounds of Formula I, or any of the specific subgroups, subclasses, or specific compounds described above, in an amount sufficient to inhibit a bitter taste. Dental bleaching compositions are known in the art. See, e.g., U.S. Pat. No. 6,485,709, which is herein incorporated by reference in its entirety. A dental bleaching composition of the present invention intended for use with dental trays can utilize a sticky carrier formed from a fluid and a thickener. The sticky carrier accordingly can comprise finely divided silica, such as silica fume, dispersed in a liquid, such as a polyol. Examples of suitable polyols include propylene glycol, glycerin, polypropylene glycols, sorbitol, polyethylene glycols and the like. While the carrier preferably includes thickeners, the carrier can also be only a liquid such as water or any of the liquid polyols without any thickeners.

Additionally, the present invention is directed to a process of preparing an improved dental hygienic composition, wherein the improvement comprises adding one or more compounds of Formula I, or any of the specific subgroups, subclasses, or specific compounds described above, to a dental bleaching composition. In certain embodiments, the one or more compounds of Formula I are added to a dental hygienic composition in an amount of about 1% to about 20%, preferably about 1% to about 5%, or about 5%, 10%, or 15%, by weight.

The present invention is also directed to a cosmetic composition comprising one or more compounds of Formula I, or any of the specific subgroups, subclasses, or specific compounds described above. For example, but not by way of limitation, the cosmetic composition comprising a compound of Formula I, or any of the specific subgroups, subclasses, or specific compounds described above, can include a face cream, lipstick, lip gloss, and the like. Other suitable compositions of the invention include lip balm, such as CHAPSTICK® or BURT'S BEESWAX® Lip Balm, further comprising one or more compounds of Formula I, or any of the specific subgroups, subclasses, or specific compounds described above.

Additionally, the invention is directed to a process of preparing an improved cosmetic composition, wherein the improvement comprises adding one or more compounds of Formula I, or any of the specific subgroups, subclasses, or specific compounds described above, to a cosmetic composition. In certain embodiments, the one or more compounds of Formula I, or any of the specific subgroups, subclasses, or specific compounds described above, are added to a cosmetic composition in an concentration of about 1% to about 20%, or about 1% to about 5%, or about 1%, 2%, or 3%, by weight, of the cosmetic composition.

The present invention is also directed to a food composition comprising one or more compounds of Formula I, or any of the specific subgroups, subclasses, or specific compounds described above. In some embodiments, the food composition is one which exhibits an undesirable taste, such as a bitter taste, which can be masked by a compound of Formula I, or any of the specific subgroups, subclasses, or specific compounds described above. Furthermore, in a preferred embodiment, the food composition comprises a compound of Formula I, or any of the specific subgroups, subclasses, or specific compounds described above in an amount sufficient to inhibit an unpleasant taste.

Specific food compositions and food ingredients to which one of more compounds of Formula I, or any of the specific subgroups, subclasses, or specific compounds described above, can be added include, but are not limited to, potassium chloride, ammonium chloride, sodium chloride (e.g., table salt), magnesium chloride, halide salts, naringin, caffeine, urea, magnesium sulfate, saccharin, acetosulfames, aspirin, potassium benzoate, potassium bicarbonate, potassium carbonate, potassium nitrate, potassium nitrite, potassium sulfate, potassium sulfite, potassium glutamate, food preservatives in their pharmaceutically acceptable salts, antibiotics, unsweetened chocolate, cocoa beans, yogurt, preservatives, flavor enhancers, dietary supplements, gelling agents, pH control agents, nutrients, processing aids, bodying agents, dispersing agents, stabilizers, colorings, coloring diluents, anticaking agents, antimicrobial agents, formulation aids, leavening agents, surface active agents, anticaking agents, nutrient supplements, alkali, acids, sequestrants, denuding agents, general purpose buffers, thickeners, cooked out juice retention agents, color fixatives in meat and meat compositions, color fixatives in poultry and poultry compositions, dough conditioners, maturing agents, yeast foods, mold retardants, emulsifiers, texturizers, binders, water correctives, miscellaneous and general purpose food additives, tabletting aids, lye peeling agents, washing water agents, oxidizers, antioxidants, enzymes, extenders, fungicides, cake mixes, coffee, tea, dry mixes, non-dairy creamers, salts, animal glue adjuvant, cheese, nuts, meat and meat compositions, poultry and poultry composition, pork and pork compositions, fish and fish compositions, vegetable and vegetable compositions, fruit and fruit compositions, smoked compositions such as meat, cheese fish, poultry, and vegetables,. whipping agents, masticatory substances in chewing gums, dough strengtheners, animal feed, poultry feed, fish feed, pork feed, defoaming agents, juices, liquors, substances or drinks containing alcohol, beverages including but not limited to alcoholic beverages and non-alcoholic carbonated and/or non-carbonated soft drinks, whipped toppings, bulking agents used in foods including but not limited to starches, corn solids, polysaccharides and other polymeric carbohydrates, icings, as well as potassium-containing or metal-containing substances with undesirable tastes and the like.

Moreover, the present invention contemplates the preparation of foods such as breads, biscuits, pancakes, cakes, pretzels, snack foods, baked goods, etc. prepared using for example potassium bicarbonate or potassium carbonate in place of the sodium salts as leavening agents in conjunction with a compound of Formula I, or any of the specific subgroups, subclasses, or specific compounds described above, in an amount sufficient to eliminate one or more undesirable tastes. The compound of Formula I, or any of the specific subgroups, subclasses, or specific compounds described above, can be typically present in a concentration of about 0.001% to about 50%, about 0.001% to about 10%, about 0.001% to about 1%, about 0.01% to about 50%, about 0.01% to about 10%, about 0.01% to about 1%, about 0.1% to about 50%, about 0.1% to about 10%, or about 0.1% to about 1% by weight, of the material with the undesirable taste. The present invention also contemplates the preparation of preservatives for foods comprising the potassium salts of benzoate, nitrate, nitrite, sulfate, and sulfite and so on, in conjunction with an appropriate concentration of a compound of Formula I, or any of the specific subgroups, subclasses, or specific compounds described above, to eliminate undesirable tastes in foodstuffs.

Thus, the invention is directed to a process of preparing an improved food composition, wherein the improvement comprises adding one or more compounds of Formula I, or any of the specific subgroups, subclasses, or specific compounds described above, to a food composition. In some embodiments, one or more compounds of Formula I are added to a food composition in an amount of about 1% to about 50%, by weight, or alternatively, about 1% to about 20%, by weight, of the food composition. In some embodiments, one or more compounds of Formula I are added to a food composition in an amount of about 0.01% to about 50%, about 0.01% to about 20%, about 0.01% to about 5%, about 0.01% to about 1%, about 0.1% to about 50%, about 0.1% to about 20%, or about 0.1% to about 5% by weight of the food composition.

The present invention is also directed to an animal food composition comprising one or more compounds of Formula I. The one or more compounds are preferably in an amount sufficient to inhibit one or more undesirable tastes associated with the animal food composition. Animal food compositions are well known in the art, see, e.g., U.S. Pat. No. 6,403,142, which is incorporated herein by reference, and include dog food, cat food, rabbit food, and the like. Animal food compositions of the present invention also include animal food compositions useful for feeding livestock, such as cattle, bison, pigs, chicken, and the like. The animal food composition of the present invention can also be a solid hypoallergenic pet food comprising a component that contains protein or protein fragments wherein all of the component is partially hydrolyzed and further comprises one or more compounds of Formula I.

Additionally, the present invention is directed to a process of preparing an improved animal food composition, wherein the improvement comprises adding one or more compounds of Formula I to an animal food composition. In some embodiments, the one or more compounds of Formula I are added to an animal food composition in an amount of 0.01% to about 50%, about 0.01% to about 20%, about 0.01% to about 5%, about 0.01% to about 1%, about 0.1% to about 50%, about 0.1% to about 20%, or about 0.1% to about 5% by weight of the animal food composition.

Additionally, any of the compositions described herein containing a compound of Formula I can further comprise one or more additional taste masking agents. Such taste masking agents include, but are not limited to, sucralose, zinc gluconate, ethyl maltol, glycine, acesulfame-k, aspartame, saccharin, fructose, xylitol, malitol, isomalt, salt, spray dried licorice root, glycyrrhizin, dextrose, sodium gluconate, sucrose, glucono-delta-lactone, ethyl vanillin, vanillin, and combinations thereof.

In some embodiments, the present invention is directed to a composition comprising a compound of Formula I and a carrier, wherein the carrier is suitable for an assay. Such carriers can include solid carriers and/or liquid carriers. In some embodiments, a composition suitable for an assay is sterile. Non-limiting examples of suitable carriers for assays include dimethylsulfoxide, ethanol, dichloromethane, methanol, and the like. In some embodiments, a composition of the present invention comprises a compound of Formula I and a carrier, wherein the compound of Formula I is in an amount suitable for inhibiting a taste modulating protein.

In each of the embodiments of the compositions described herein, a compound of Formula I can be used in varying amounts such that its concentration relative to an agent that is believed to cause the unwanted taste, such as a bitter taste. For example, a composition of the present invention can comprise or administer a compound of Formula I in a molar ratio of about 1000:1 to about 1:1000, or alternatively a molar ratio of about 500:1, about 200:1, about 10:1, about 1:1, about 1:10, about 1:200, or about 1:500, relative to an agent that is believed to cause an undesirable taste, such as a bitter taste. In another example, the present invention is directed to a food composition comprising one or more food ingredients and a compound of Formula I, wherein the molar ratio of the compound of Formula I to the food agent that causes, or is believed to cause, a bitter taste is about 1000:1 to about 1:1000, or alternatively, about 500:1, about 200:1, about 10:1, about 1:1, about 1:10, about 1:200, or about 1:500. As will be appreciated, the various ranges and amounts of the compounds of Formula I can be used with modifications, if preferred, in each of the embodiments described herein.

The activity of a compound of Formula I, or any of the specific subgroups, subclasses, or specific compounds described above can be determined by testing the compound using a number of methods known in the art. For example, the effectiveness or ability of a compound to mask a bitter taste can be assessed by performing an in vivo taste assay. Such an in vivo assay can be performed by testing the activity of the compounds using human subjects that are further administered bitter tasting compound. A solution having a concentration of a bitter tasting compound, such as, for example quinine in water, is first found that the subject rates as 5 for bitterness on a scale of 0 to 10, wherein 0 is no bitterness and 10 is the most intense bitterness the subject has ever encountered. To the solution having a concentration of quinine in water with a bitterness value of 5, an amount of one or more compounds of Formula I, as described above, is then added, and the resulting solution is administered to the subject. The subject then rates the bitterness of this solution on the same scale, and the concentration of the bitterness-masking compound according to Formula I can be determined, which is required to give the desired taste masking effect.

The activity of a compound of Formula I, or any of the specific subgroups, subclasses, or specific compounds described above, can also be determined by means of the assay described in Example 11. The assay is described in complete detail in co-pending application Ser. No. 60/732,636, which is incorporated by reference herein in its entirety.

Methods of Preparing the Compounds

A compound of Formula I can be synthesized by methods described below and/or using procedures known in the art. The compounds for use in the present invention can be synthesized using procedures known in the art. See, for example, J. Indian Chem. Soc. 73(6):283-284 (1996); Bioorg. Med. Chem. Lett. 5(11):1171-1176 (1995); Chem. Lett., 12:1457-1460 (1977); and Tet. Lett. 61:3539-3546 (2005), which are incorporated herein by reference in their entirety.

The following general schemes illustrate synthetic methods used to prepare compounds of the present invention.

A compound of Formula II can be prepared by condensation of an aromatic aldehyde with a diketone or an α-hydroxy ketone in the presence of ammonium acetate and 1-butyl imidazolium tetrafluoroborate at approximately 100° C. In some embodiments, such a reaction can be carried out neat (e.g., without a solvent).

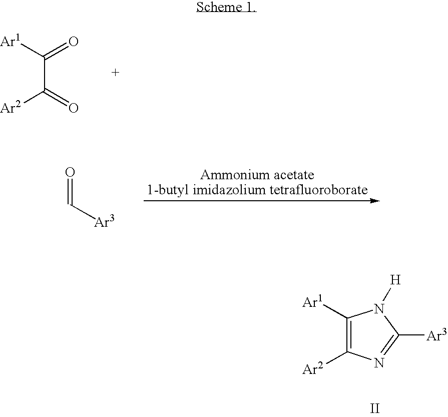

After the reaction is complete, a compound of Formula II can be isolated by, for example, crystallization in ethanol, dichloromethane, ethyl acetate, toluene, and the like.

A compound of Formula I can be prepared from compounds of Formula II by treatment with an arylhalide of Formula R¹—X in the presence of a base (e.g., K₂CO₃, Cs₂CO₃, etc.) and a polar aprotic solvent (e.g., acetone, acetonitrile, dimethylsufloxide, etc.), and where R¹ has been defined previously, and X is Fluorine, Chlorine, Bromine or Iodine. After the reaction is complete, the reaction mixture is diluted with solvent, washed with water, and dried. After drying, the solvent is evaporated to give a compound of Formula I.

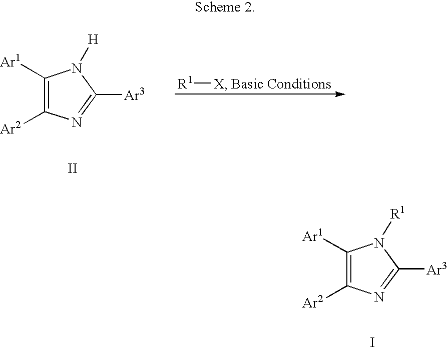

After the reaction is complete, the composition can be isolated by crystallization from solvents such as ethanol, dichloromethane, ethyl acetate, and toluene.

Similarly, other compounds of the present invention can be obtained and isolated from commercial sources, or prepared by persons of ordinary skill in the art. Starting materials are commercially available or they can be prepared by persons of ordinary skill in the art.

Of course, other methods and procedures known in the art can be used to prepare certain compounds of Formula I.

The following examples are illustrative, but not limiting, of the method, compounds, and compositions of the present invention. Each of the compounds listed below was obtained from commercially available catalogs from companies such as Aldrich RareChemLib, Aldrich Sigma, AsInEx, Biotech Corp., Brandon/Berlex, Calbiochem, ChemBridge, Comgenex West, G. & J. Research, IBS, ICN Biochemicals, Institute for Chemotherapy, Kodak, Lederle Labs, Ligand-CGX, Maybridge_PRI, Menai Organics, MicroSource, MPI Chemists, Mybrdg/ONYX, Receptor Research, RGI, Rhone-Poulenc, SPECS/BioSPECS, SYNTHESIA, T. Glinka, Tripos Modern, VWR, Zaleska, and Zelinsky/Berlex. The compounds were purified using conventional purification procedures, such as HPLC. The identity of each compound was confirmed using HPLC and mass spectrometry. Suitable modifications and adaptations, obvious to those skilled in the art, of the conditions and parameters used to produce or purify the compounds are within the spirit and scope of the invention.

### EXAMPLES

Compounds of Examples 1-10 are prepared and re-crystallized according to the reaction Schemes 1 and 2, above.

### Example 1

### methyl4-(4,5-diphenyl-1H-imidazol-2-yl)benzoate

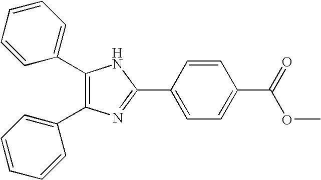

Molecular Formula: C₂₃H₁₈N₂O₂; Molecular Weight: 354 (calculated), 355 (found).

### Example 2

### [4-(4,5-diphenyl-1H-imidazol-2-yl)phenyl]methanol

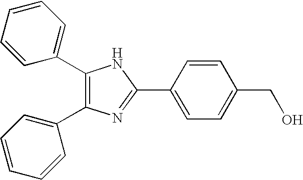

Molecular Formula: C₂₂H₁₈N₂O; Molecular Weight: 326 (calculated), 327 (found); Melting Point: 50° C.

### Example 3

### 4-(4,5-diphenyl-1H-imidazol-2-yl)aniline

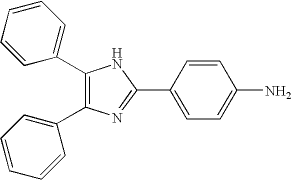

Molecular Formula: C₂₁H₁₇N₃; Molecular Weight: 311 (calculated), 312 (found).

### Example 4

### 4-(4,5-diphenyl-1H-imidazol-2-yl)phenol

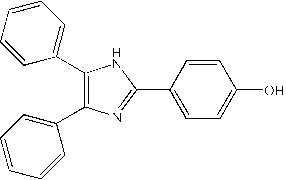

Molecular Formula: C₂₁H₁₆N₂O; Molecular Weight: 312 (calculated), 313 (found).

### Example 5

### methyl4-(4,5-diphenyl-1H-imidazol-2-yl)phenylcarbamate

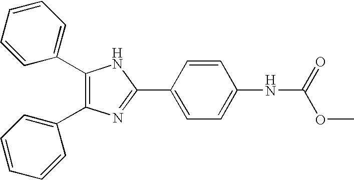

Molecular Formula: C₂₃H₁₉N₃O₂; Molecular Weight: 369 (calculated), 368 (found).

### Example 6

### N-[4-(4,5-diphenyl-1H-imidazol-2-yl)phenyl]acetamide

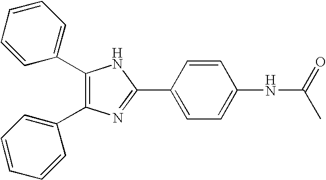

Molecular Formula: C₂₃H₉N₃O; Molecular Weight: 353 (calculated), 354 (found).

### Example 7

### 4-(4,5-diphenyl-1H-imidazol-2-yl)benzonitrile

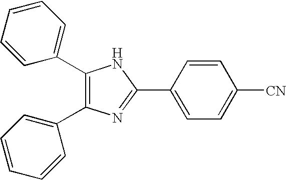

Molecular Formula: C₂₂H₁₅N₃; Molecular Weight: 321 (calculated), 322 (found).

### Example 8

### N-[4-(4,5-diphenyl-1H-imidazol-2-yl)benzyl]methanesulfonamide

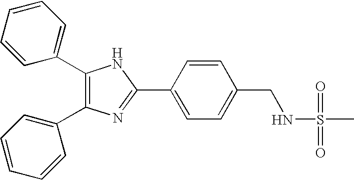

Molecular Formula: C₂₃H₂₁N₃SO₂; Molecular Weight: 403 (calculated), 404 (found).

### Example 9

### 4-(4,5-diphenyl-1H-imidazol-2-yl)benzoic acid

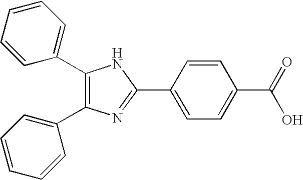

Molecular Formula: C₂₂H₁₆N₂O₂; Molecular Weight: 340 (calculated), 341 (found).

### Example 10

### 2-(4-nitro-phenyl)-4,5-diphenyl-1H-imidazole

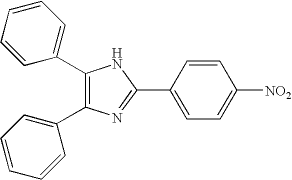

Molecular Formula: C₂₁H₁₅N₃O₂; Molecular Weight: 341 (calculated), 342 (found).

### Example 11

### Activity of Selected Compounds

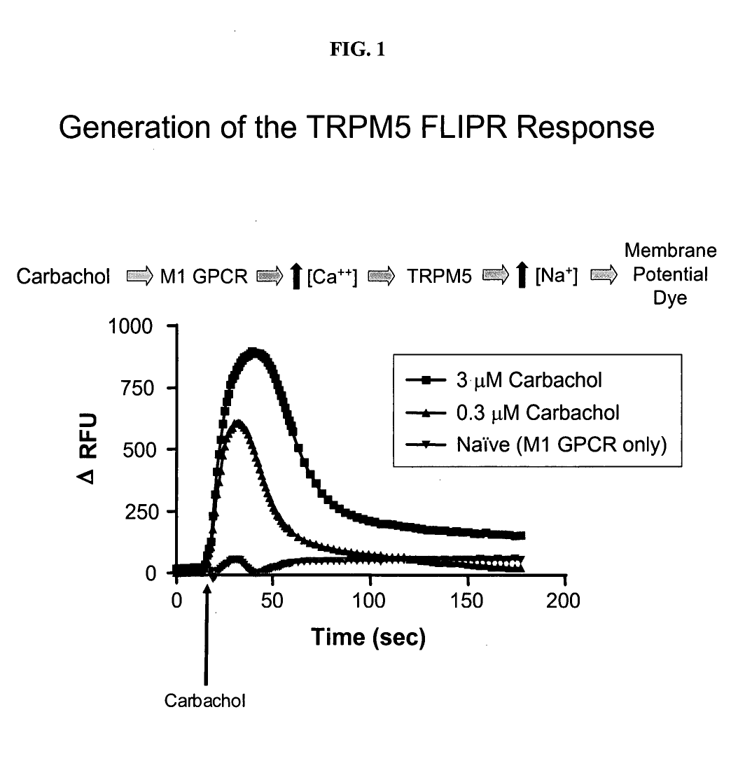

Modulation of the activity of human taste receptor proteins was measured in live cells on a fluorescent imaging plate reader (FLIPR). The basis of the assay (shown in FIG. 1) is the calcium-dependent activation of the ion channel which occurs via by activation of a G-protein coupled receptor (GPCR). GPCR activation by an appropriate agonist causes a transient increase in intercellular Ca²⁺ ion concentration which in turn causes the ion channel to open, allowing an influx of Na⁺ ions. This influx leads to a change in the membrane potential of the cell, which can be monitored as a change in the fluorescent signal from voltage-dependent (membrane potential) fluorescent dyes.

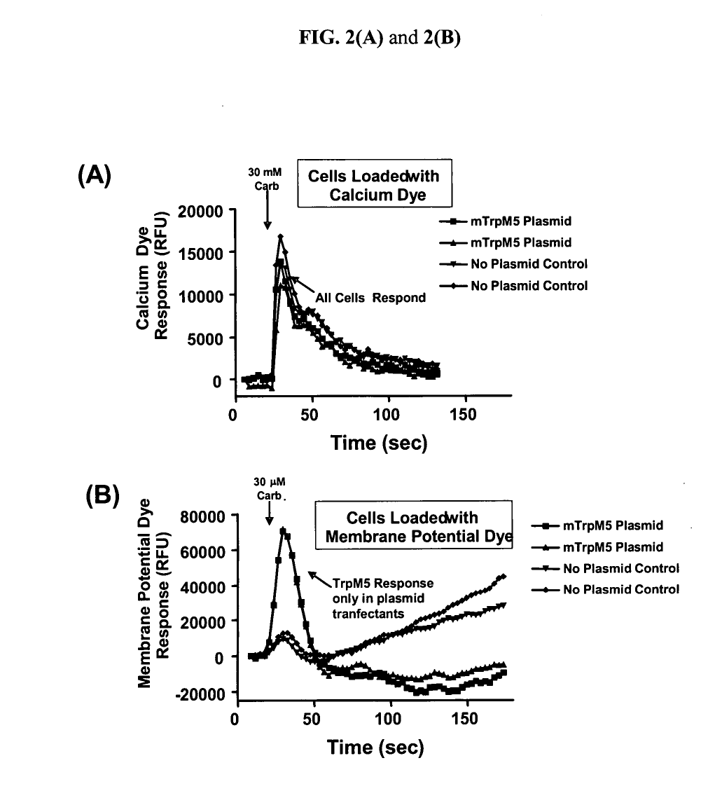

A schematic representation of the assay is shown in FIGS. 2(A) and 2(B), where traces of fluorescent response versus time are shown for cells transfected with a plasmid encoding human TRPM5 or cells containing sham plasmid controls. While all cells had a Ca²⁺ response to the endogenous muscarinic GPCR agonist carbachol (excitation at 485 nm, and emission detected at 525 nm), as shown in FIG. 2(A), only cells containing the taste-modulating protein plasmid showed a sharp peak for the membrane potential dye response (excitation at 530 nm, and emission detected at 565 nm), as shown in FIG. 2(B).

For the screening assay, the human TRPM5 gene was cloned, HEK293 cells were transfected with a plasmid containing the gene, and a stable, high expression clone was chosen for the screen. Cells were grown in standard media at 37° C. The day before screening, the cells were removed from flasks and added to 384 well clear bottom plates (12K cells in 20 μL/well). On the assay day, 20 μL of membrane potential dye (Part No. R8123, Molecular Devices Corp.) was added to the cells and dye was allowed to be taken up, i.e., loaded, into the cells for 1 hr at 37° C. The dye-loaded cell plate was placed in the FLIPR along with a second 384 well plate containing test compounds as well as positive (fully inhibited) and negative (non-inhibited) controls. The assay was started by addition of 10 μL of solution from the compound plate into the cell plate. During this process, continuous fluorescent recordings were made simultaneously for all wells. After addition of the compound solution, the tips were automatically washed and 10 μL of a stimulation solution of 60 μM ATP (an agonist for an endogenous purinurgic GPCR), was added to all wells of the cell plate (to give a final concentration of 10 μM ATP). The height of the response was calculated and percent inhibition values, versus negative control wells, was calculated for the test samples.

Two counterscreen assays were run on separate cell plates utilizing the same cells as described above. In the calcium counterscreen, the cells were loaded with a calcium sensitive dye (Calcium3 Dye, Part no. 8090, Molecular Devices Corp.) and stimulated by ATP to check for compounds that block the GPCR-mediated calcium activation step. In the KCl counterscreen, cells were stimulated with 120 mM KCl (to give a final concentration of 20 mM KCl) instead of ATP to check for compounds that inhibit the membrane potential response by virtue of being non-specific ion channel blockers.

Unless otherwise indicated, the data in Table 1 were determined using the assays described above, providing maximum percent inhibition data at concentrations between 10 μM and 100 μM.

TABLE 1

| Example No. | TRPM5 Activity | Calcium Counterscreen |
|---|---|---|
| 1 | 79 | 0 |
| 2 | 50 | 0 |
| 3 | 42 | 0 |
| 4 | 51 | 0 |
| 5 | 19 | 0 |
| 6 | 29 | 0 |
| 7 | 0 | 0 |
| 8 | 90 | 0 |
| 9 | 60 | 0 |
| 10 | 100 | 0 |

### Example 12

### Electrophysiological Test

Standard whole-cell recordings were obtained from HEK stably transfected with human TRPM5. External solution was Hank's Balanced Salt Solution (Gibco) with 20 mM 4-(2-hydroxyethyl)-1-piperazineethanesulfonic acid (HEPES) buffer, adjusted to pH 7.4. Internal solution contained 135 mM Cesium glutamate, 10 mM HEPES, 8 mM NaCl, 2 mM MgATP and 10 mM EGTA. Free calcium ion concentration [Ca²⁺] was adjusted by addition of CaCl₂, as calculated by WebMaxC program. Currents were recorded with a Multiclamp 700B amplifier (MDC) using PClamp software, filtered at 1 kHz and sampling at 5 kHz. Holding potential was −80 mV. TRPM5 current was activated by intracellular dialysis with free calcium (10⁻⁷ to 10⁻⁵ M) and sampling was done with 200 ms ramps from −80 to +80 mV at 1 Hz. Current amplitudes were measured at −80 and +80 mV and plotted versus time. Large >5 nA current (+80 mV) was activated by calcium, and no significant current was seen in non-transfected, sham HEK cells. A >90% inhibition of TRPM5 current was found when TRPM5 transfected cells were pre-treated with 10 mM of Example 10.

Having now fully described this invention, it will be understood by those of ordinary skill in the art that the same can be performed within a wide and equivalent range of conditions, formulations and other parameters without affecting the scope of the invention or any embodiment thereof. All patents and publications cited herein are fully incorporated by reference herein in their entirety.

1. A method of inhibiting a taste modulating protein, the method comprising contacting the taste modulating protein with a compound of Formula I: 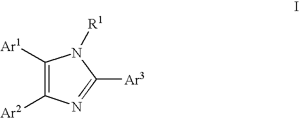
or a pharmaceutically acceptable salt thereof; wherein:
R¹ is hydrogen, unsubstituted alkyl, or unsubstituted arylalkyl;
Ar¹ and Ar² are independently phenyl or heteroaryl, either of which is optionally substituted; and
Ar³ is an optionally substituted: phenyl or heteroaryl.

2. The method of claim 1, wherein Ar¹ and Ar² are independently selected from the group consisting of optionally substituted: pyridyl, pyrimidinyl, pyridazinyl, pyrimidinyl, pyrazinyl, and triazinyl, and N-oxides thereof.

3. The method of claim 1, wherein Ar¹ and Ar² are independently selected from the group consisting of: unsubstituted phenyl, methylphenyl, methoxyphenyl, halophenyl, cyanophenyl, carboxyphenyl, aminophenyl, and hydroxyphenyl.

4. (canceled)

5. (canceled)

6. The method of claim 1, wherein Ar³ is selected from the group consisting of optionally substituted phenyl, and optionally substituted: pyridyl, pyrimidinyl, pyridazinyl, pyrimidinyl, pyrazinyl, and triazinyl, and N-oxides thereof.

7. (canceled)

8. (canceled)

9. The method of claim 1, wherein Ar³ is selected from the group consisting of: phenyl, C-attached pyridyl, C-attached pyrimidyl, and C-attached pyridazinyl; any of which is optionally substituted with one or more: nitro, halo, cyano, carboxyl, amino, hydroxyl, alkyl, and cyanoalkyl substituents in any one or more of the ortho-, meta-, and para-positions.

10. The method of claim 1, wherein
R¹ is hydrogen, unsubstituted C₁₋₄ alkyl, or unsubstituted aryl(C₁₋₄)alkyl;
Ar¹ and Ar² are both unsubstituted phenyl; and
Ar³ is phenyl, optionally substituted by one to three substituents independently selected from the group consisting of carboxy, alkoxycarbonyl, hydroxy, hydroxyalkyl, amino, alkoxycarbonylamino, cyano, alkylsulfonylaminoalkyl, and nitro.

11. The method of claim 10, wherein Ar³ is phenyl, substituted in the para-position by one carboxy, alkoxycarbonyl, hydroxyalkyl, hydroxy, amino, alkoxycarbonylamino, cyano, alkylsulfonylaminoalkyl, or nitro.

12. The method of claim 1, wherein the compound of Formula I is one of:
methyl4-(4,5-diphenyl-1H-imidazol-2-yl)benzoate;
[4-(4,5-diphenyl-1H-imidazol-2-yl)phenyl]methanol;
4-(4,5-diphenyl-1H-imidazol-2-yl)aniline;
4-(4,5-diphenyl-1H-imidazol-2-yl)phenol;
methyl4-(4,5-diphenyl-1H-imidazol-2-yl)phenylcarbamate;
N-[4-(4,5-diphenyl-1H-imidazol-2-yl)phenyl]acetamide;
4-(4,5-diphenyl-1H-imidazol-2-yl)benzonitrile;
N-[4-(4,5-diphenyl-1H-imidazol-2-yl)benzyl]methanesulfonamide;
4-(4,5-diphenyl-1H-imidazol-2-yl)benzoic acid; and
2-(4-nitro-phenyl)-4,5-diphenyl-1H-imidazole;
or a pharmaceutically acceptable salt thereof.

13. The method of claim 1, wherein the taste modulating protein is a non-human TRPM5 protein.

14. (canceled)

15. The method of claim 1, wherein the taste modulating protein is a human TRPM5 protein.

16. (canceled)

17. The method of claim 1, wherein the compound of Formula I is administered as a pharmaceutical, veterinary, food, cosmetic, or dental hygeinic composition.

18. The method of claim 17, wherein the compound of Formula I is administered in a concentration of about 0.01% to about 50%, by weight, of the composition.

19-26. (canceled)

27. The method of claim 1, wherein the compound of Formula I is administered in an amount of about 0.01 mg to about 100 mg.

28. The method of claim 1, wherein the compound of Formula I is administered in an amount sufficient to inhibit the taste-modulating protein by about 10% to about 95%.

29-53. (canceled)

54. A method of inhibiting the depolarization of a taste receptor cell, comprising contacting the taste receptor cell with one or more compounds of Formula I: 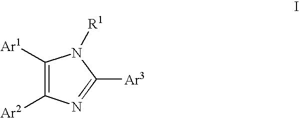
or a pharmaceutically acceptable salt thereof; wherein:
R¹ is hydrogen, unsubstituted alkyl, or unsubstituted arylalkyl;
Ar¹ and Ar² are independently phenyl or heteroaryl, either of which is optionally substituted; and
Ar³ is an optionally substituted: phenyl or heteroaryl.

55. The method of claim 54, wherein Ar¹ and Ar² are independently selected from the group consisting of optionally substituted phenyl, and optionally substituted pyridyl, pyrimidinyl, pyridazinyl, pyrimidinyl, pyrazinyl, and triazinyl, and N-oxides thereof.

56. The method of claim 54, wherein Ar¹ and Ar² are independently selected from the group consisting of: unsubstituted phenyl, methylphenyl, methoxyphenyl, halophenyl, cyanophenyl, carboxyphenyl, aminophenyl, and hydroxyphenyl.

57. (canceled)

58. (canceled)

59. The method of claim 54, wherein Ar³ is selected from the group consisting of an optionally substituted: phenyl, pyridyl, pyrimidinyl, pyridazinyl, pyrimidinyl, pyrazinyl, and triazinyl.

60. (canceled)

61. (canceled)

62. The method of claim 54, wherein Ar³ is selected from the group consisting of phenyl, C-attached pyridyl, C-attached pyrimidyl, and C-attached pyridazinyl, any of which is optionally substituted with one or more nitro, halo, cyano, carboxyl, amino, hydroxyl, alkyl, and cyanoalkyl substituents in any one or more of the ortho-, meta-, and para-positions.

63. The method of claim 54, wherein:
R¹ is hydrogen, unsubstituted C₁₋₄ alkyl, or unsubstituted aryl(C₁₋₄)alkyl;
Ar¹ and Ar² are both unsubstituted phenyl; and
Ar³ is phenyl, substituted by one to three substituents independently selected from the group consisting of carboxy, alkoxycarbonyl, hydroxy, hydroxyalkyl, amino, alkoxycarbonylamino, cyano, alkylsulfonylaminoalkyl, and nitro.

64. The method of claim 63, wherein Ar³ is phenyl, substituted in the para-position by one carboxy, alkoxycarbonyl, hydroxyalkyl, hydroxy, amino, alkoxycarbonylamino, cyano, alkylsulfonylaminoalkyl, or nitro.

65. The method of claim 54, wherein the compound of Formula I is one of:
methyl4-(4,5-diphenyl-1H-imidazol-2-yl)benzoate;
[4-(4,5-diphenyl-1H-imidazol-2-yl)phenyl]methanol;
4-(4,5-diphenyl-1H-imidazol-2-yl)aniline;
4-(4,5-diphenyl-1H-imidazol-2-yl)phenol;
methyl4-(4,5-diphenyl-1H-imidazol-2-yl)phenylcarbamate;
N-[4-(4,5-diphenyl-1H-imidazol-2-yl)phenyl]acetamide;
4-(4,5-diphenyl-1H-imidazol-2-yl)benzonitrile;
N-[4-(4,5-diphenyl-1H-imidazol-2-yl)benzyl]methanesulfonamide;
4-(4,5-diphenyl-1H-imidazol-2-yl)benzoic acid; and
2-(4-nitro-phenyl)-4,5-diphenyl-1H-imidazole;
or a pharmaceutically acceptable salt thereof.

66. The method of claim 54, wherein the taste receptor cell is human.

67. The method of claim 54, wherein the compound of Formula I is administered as a pharmaceutical, veterinary, food, cosmetic, or dental hygienic composition.

68. The method of claim 67, wherein the compound of Formula I is administered in a concentration of about 0.01% to about 50%, by weight, of the composition.

69. The method of claim 54, wherein the compound of Formula I is administered in an amount of about 0.01 mg to about 100 mg.

70. The method of claim 54, wherein the compound of Formula I is administered in an amount sufficient to inhibit the depolarization of the taste-receptor cell by about 10% to about 95%.

71. The method of claim 54, wherein the taste receptor cell can sense a taste produced by a biologically active agent.

72. The method of claim 71, wherein the taste receptor cell can sense a taste produced by a biologically active agent selected from the group consisting of analgesics, anesthetics, anorexiants, appetite depressants, antacidics, antiasthmatics, antidiuretics, antipyretics, antihistamines, anticholinergics, antidiarrheals, antitussives, antinauseants, antiarrhythmics, antimicrobials, antibacterials, antifungals, antivirals, anti-inflammatory agents, agents active against flatulence, antimigraine agents, beta-receptor blockers, bronchodilators, psychopharmacological agents, spasmolytics, sedatives, antihyperkinetics, tranquilizers, decongestants, demulcents, agents for alcohol withdrawal, antitussives, fluorine supplements, laxatives, local antibiotics, corticosteroid supplements, agents against goiter formation, antiepileptics, agents against dehydration, antiseptics, NSAIDs, H₂-receptor antagonists, nutritional supplements, gastrointestinal active agents, alkaloids, supplements for trace elements, ion-exchange resins, cholesterol-depressant agents, lipid-lowering agents, expectorants, and combinations thereof.

73. The method of claim 54, wherein the taste receptor cell can sense a bitter taste.

74-213. (canceled)

214. The method of claim 54, wherein the compound of Formula I is administered in an amount sufficient to inhibit the depolarization of the taste-receptor cell by about 25% to about 80%.
